(19) United States
(12) Patent Application Publication
(10) Pub. No.: US 2020/0367862 A1
(43) Pub. Date: Nov. 26, 2020

(54) ULTRASOUND DIAGNOSTIC DEVICE AND ULTRASOUND DIAGNOSTIC DEVICE CONTROL METHOD

(71) Applicant: Konica Minolta, Inc., Tokyo (JP)

(72) Inventor: Tetsuya TANIGUCHI, Tokyo (JP)

(21) Appl. No.: 16/875,438

(22) Filed: May 15, 2020

(30) Foreign Application Priority Data
May 20, 2019 (JP) 2019-094651
Mar. 27, 2020 (JP) 2020-058170

(51) Int. Cl. A61B 8/00; A61B 8/08; G01S 7/52

(52) U.S. Cl. CPC A61B 8/54; G01S 7/5209; A61B 8/5207; A61B 8/4488

(57) ABSTRACT

An ultrasound diagnostic device including a transmitter and a delay-and-sum unit. The transmitter selects a tertiary transducer array that coincides in an azimuth direction with a transmission focal point, two partial primary transducer arrays that sandwich the tertiary transducer array in the azimuth direction, and two secondary transducer arrays that sandwich the tertiary and partial primary transducer arrays in the azimuth direction, and causes transmission from the tertiary and secondary transducer arrays of an ultrasound beam with a larger signal intensity in a high frequency band than that transmitted from the partial primary transducer arrays. The delay-and-sum unit sets calculation target areas that have different positions in the azimuth direction, and executes delay-and-sum processing with respect to each of the calculation target areas to generate acoustic line signal frame data.

This application claims priority to Japanese Patent Application No. 2019-094651 filed May 20, 2019 and Japanese Patent Application No. 2020-058170 filed Mar. 27, 2020, the contents of which are hereby incorporated by reference in their entirety.

### BACKGROUND OF THE DISCLOSURE

### Technical Field

The present disclosure relates to ultrasound diagnostic devices and ultrasound diagnostic device control methods, and in particular to beamforming methods pertaining to transmission and reception of ultrasound diagnostic devices.

### Description of the Related Art

An ultrasound diagnostic device is a medical imaging device that acquires in-vivo information through an ultrasound pulse reflection method and displays the information as a tomographic image. An ultrasound diagnostic device transmits an ultrasound wave into a subject via an ultrasound probe (also referred to as a probe), receives an ultrasound reflected wave (echo) generated due to a difference in acoustic impedance of tissue in the subject, then based on an electrical signal obtained from this reception, generates an ultrasound tomographic image showing structure of the tissue in the subject, and displays the ultrasound tomographic image on a monitor (also referred as a display). Compared to other modalities that use X-rays or radiation, ultrasound diagnostic devices are widely used for morphological diagnoses of living organisms because they allow observation of states of internal tissue in real time by using tomographic images and the like.

Various proposals have been made to improve real-time performance of ultrasound diagnostic devices, for example use of a technique of simultaneously transmitting and receiving in two directions from the same aperture has been proposed, as in JP 2002-336246. As another method, a technique has been proposed in which transducers are divided into a plurality of areas in order to simultaneously transmit in two directions, as in JP 2010-22654. With these methods, the time required for transmission and reception can be reduced by half, and real-time performance can be improved accordingly.

### SUMMARY

An object of the present disclosure is to provide an ultrasound diagnostic device and a control method that improve visibility of anisotropic highly reflective members in a peripheral region of a shallow region of an ultrasound irradiation region and improve visualization of a high-angle anisotropic reflective site, without impairing real-time performance, without requiring complex transmission control, and without greatly increasing an amount of heat generated by a probe due to transmission, even in an inexpensive device.

According to an embodiment of the present disclosure, the ultrasound diagnostic device is an ultrasound diagnostic device that transmits an ultrasound beam into a subject using an ultrasound probe in which transducers are arrayed along an azimuth direction, and generates acoustic line signals based on reflected waves obtained from the subject, the ultrasound diagnostic device comprising: ultrasound signal processing circuitry, the ultrasound signal processing circuitry comprising: a transmitter that determines a transmission focal point corresponding to an ultrasound beam focal point, selects an array of transmission transducers from the transducers, and causes transmission of an ultrasound beam focused on the transmission focal point from the array of transmission transducers; an input unit that generates sequences of received signals corresponding one-to-one with reception transducers in an array selected from the transducers, based on reflected waves received by the array of reception transducers; a delay-and-sum unit that determines, from analysis target areas in the subject, calculation target areas that partially overlap each other, selects a reception aperture transducer array from the reception transducers, and with respect to observation points in the calculation target areas, executes delay-and-sum processing of the received signal sequences corresponding one-to-one with the reception transducers included in the reception aperture; and an imaging signal synthesizer that synthesizes results of the delay-and-sum processing using positions of the observation points for reference to generate ultrasound imaging signal frame data, wherein the transmitter selects, as the array of transmission transducers, a primary transducer array and two secondary transducer arrays that sandwich the primary transducer array in the azimuth direction, a portion of the ultrasound beam from the secondary transducer arrays has a larger signal intensity in a high frequency band than a portion of the ultrasound beam from the primary transducer array, and the calculation target areas each have a different position along the azimuth direction.

### BRIEF DESCRIPTION OF DRAWINGS

These and other objects, advantages, and features of the technology pertaining to the present disclosure will become apparent from the following description thereof taken in conjunction with the accompanying drawings, which illustrate at least one embodiment of the technology pertaining to the present disclosure.

FIG. 1 is a perspective view diagram of an ultrasound diagnostic system 1000 including an ultrasound diagnostic device 100 pertaining to Embodiment 1.

FIG. 2 is a function block diagram illustrating structure of the ultrasound diagnostic device 100.

FIG. 3 is a function block diagram illustrating structure of a transmitter 103 of the ultrasound diagnostic device 100.

FIG. 4A, 4B, 4C are schematic diagrams illustrating drive pulse signals. FIG. 4A and FIG. 4B illustrate an example sp of a drive pulse signal generated by the transmitter 103, and FIG. 4C illustrates an example sc of a drive pulse signal according to a different scheme.

FIG. 5 is a schematic diagram illustrating propagation paths of an ultrasound beam transmitted according to the transmitter 103.

FIG. 6 is a diagram illustrating a relationship between depth of a transmission focal point and a drive signal of a transmission transducer, with respect to an ultrasound beam transmitted according to the transmitter 103.

FIG. 7A, 7B, 7C are schematic diagrams illustrating frequency distributions of ultrasound beams transmitted according to the transmitter 103.

FIG. 8 is a function block diagram illustrating structure of a receiver 104 of the ultrasound diagnostic device 100.

FIG. 9A, 9B, 9C are schematic diagrams for explanation of acoustic line signal generation for observation points Pij by a delay-and-sum unit 1043.

FIG. 10A, 10B, 10C are schematic diagrams for explanation of acoustic line signal generation for observation points Pij by the delay-and-sum unit 1043 when a transmission steering angle θT is added.

FIG. 11 is a diagram illustrating a relationship between display depth and overall image quality in an ultrasound image generated by an ultrasound imaging signal generator 105.

FIG. 12 is a schematic diagram for explaining an example of generation of ultrasound image frame data by a synthesizer 106.

FIG. 13 is a flowchart illustrating an overview of processing by the ultrasound diagnostic device 100.

FIG. 14 is a flowchart illustrating details of transmission and reception beamforming (step S20 of FIG. 13).

FIG. 15 is a flowchart illustrating details of transmission and reception beamforming (step S20 of FIG. 13).

FIG. 16 is a schematic diagram illustrating a change in sub-scanning of an ultrasound beam propagation path pertaining to transmission according to the transmitter 103.

FIG. 17 is a flowchart illustrating processing by the ultrasound diagnostic device 100.

FIG. 18A, 18B, 18C are schematic diagrams illustrating ultrasound beam propagation paths pertaining to transmission according to the transmitter 103 of an ultrasound diagnostic device pertaining to Modification 1.

FIG. 19A, 19B, 19C, 19D are schematic diagrams for explaining generation of ultrasound image frame data by the synthesizer 106 of an ultrasound diagnostic device pertaining to Modification 1.

FIG. 20 is a flowchart illustrating processing by the ultrasound diagnostic device pertaining to Modification 1.

FIG. 21A, 21B, 21C are schematic diagrams for explaining acoustic line signal generation for observation points Pij by the delay-and-sum unit 1043 of an ultrasound diagnostic device pertaining to Embodiment 2.

FIG. 22A, 22B, 22C are schematic diagrams for explaining acoustic line signal generation for observation points Pij by the delay-and-sum unit 1043 when a transmission steering angle θT is added, with respect to the ultrasound diagnostic device pertaining to Embodiment 2.

FIG. 23 is a schematic diagram illustrating ultrasound beam propagation paths pertaining to transmission according to the transmitter 103 of an ultrasound diagnostic device pertaining to Modification 2.

FIG. 24 is a schematic diagram illustrating ultrasound beam propagation paths pertaining to transmission according to the transmitter 103 of an ultrasound diagnostic device pertaining to Modification 3.

FIG. 25 is a diagram illustrating a relationship between depth of a transmission focal point and a drive signal of a transmission transducer, with respect to an ultrasound beam transmitted according to the transmitter 103 of an ultrasound diagnostic device pertaining to Modification 3.

FIG. 26A, 26B, 26C, 26D are schematic diagrams illustrating ultrasound beam frequency distributions pertaining to transmission according to the transmitter 103 of an ultrasound diagnostic device pertaining to Modification 3.

FIG. 27 is a schematic diagram for explaining attenuation of an ultrasound beam UsO3 transmitted from an array Tx3 according to the transmitter 103 of an ultrasound diagnostic device pertaining to Modification 3.

FIG. 28A, 28B are schematic diagrams illustrating ultrasound beam propagation paths pertaining to transmission according to the transmitter 103 of an ultrasound diagnostic device pertaining to the present disclosure, when a transmission focal point depth is less than a defined value.

FIG. 29 is a schematic diagram illustrating ultrasound beam propagation paths pertaining to transmission according to the transmitter 103 of an ultrasound diagnostic device pertaining to Modification 4.

FIG. 30 is a schematic diagram illustrating ultrasound beam propagation paths pertaining to transmission according to the transmitter 103 of an ultrasound diagnostic device pertaining to Modification 5.

### DETAILED DESCRIPTION

According to a technique described in JP 2002-336246, there is no problem with transducers that do not temporally overlap when outputting an A-type pulse for forming a beam A and a B-type pulse for forming a beam B, but it is necessary to output a common pulse that is different from the A-type pulse and the B-type pulse from transducers that temporally overlap. Depending on the time overlap of A-type and B-type, transmitted ultrasound waves that cannot be transmitted in a transmission band of a probe are required, and therefore in addition to both beam A and beam B being disturbed, when the origin of transmission and reception acoustic lines is the same, in a shallow region, a combined wavefront of beam A and a combined wavefront of beam B are formed close to each other, and therefore there is a problem in that beams interfere with each other as acoustic noise, lowering image quality.

On the other hand, according to a method described in JP 2010-22654, origin points of transmission and reception acoustic lines are far apart, making it more unlikely that they be affected by acoustic noise, but the number of elements that can be used for a maximum transmission aperture is the total number of elements/area of the probe and it is difficult to form a focal point in a deeper region. Further, if the number of system channels is smaller than the total number of elements of the probe, the maximum transmission aperture is the number of system channels/area, making it even more difficult to form a focal point in a deeper region, and therefore it is difficult to adopt this method in an inexpensive system with a small number of system channels. In addition, the number of transducers driven per unit time increases, and therefore the amount of heat generated by the probe increases, and in many cases the transmission voltage must be reduced due to surface temperature regulation, leading to a problem that even if real-time performance is improved, the signal to noise ratio of obtained images is reduced.

While the demand for real-time performance has increased, in recent years the use of ultrasound diagnostic devices in regions of orthopedic surgery, with numerous tendons and ligaments, has also increased. In some cases, reflected waves from anisotropic high-reflection members/sites located in a shallow regions or a peripheral region thereof such as high angle puncture needle axes, longitudinal boundaries, anterior talofibular ligaments, and the like are insufficiently received. Thus, there is a demand for improvement in visibility of anisotropic high-reflection members/sites in shallow peripheral regions.

### Embodiment 1

### <Structure of Ultrasound Diagnostic System 1000>

The following is a description of an ultrasound diagnostic device 100 pertaining to Embodiment 1, described with reference to the drawings.

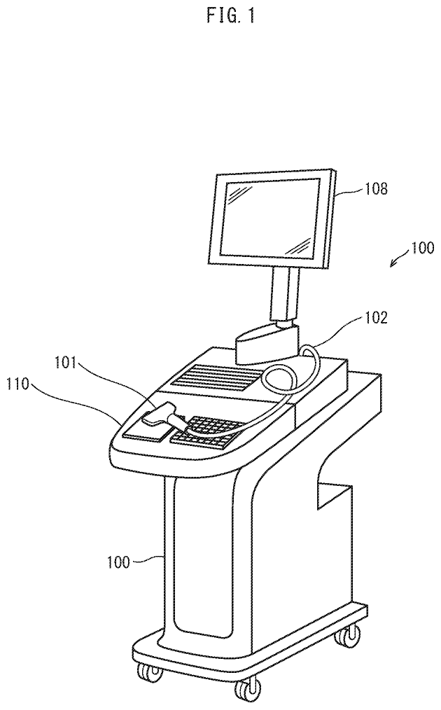

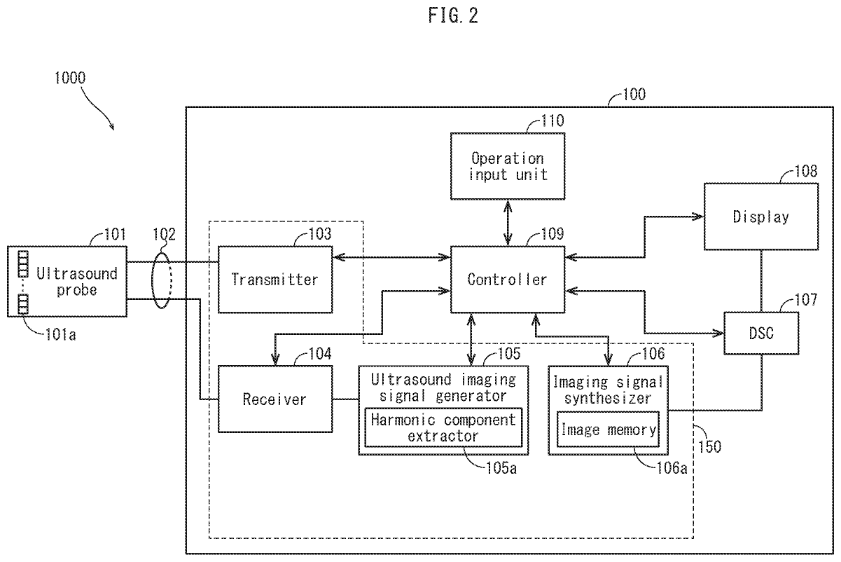

FIG. 1 is a perspective view diagram of an ultrasound diagnostic system 1000 including the ultrasound diagnostic device 100 pertaining to Embodiment 1. FIG. 2 is a function block diagram illustrating structure of the ultrasound diagnostic device 100. As illustrated in FIG. 1, the ultrasound diagnostic system 1000 includes: a probe 101 that includes a plurality of transducers 101a arrayed on a distal end surface of the probe 101 for transmitting ultrasound towards a subject and receiving resultant reflected waves; the ultrasound diagnostic device 100 that causes the probe 101 to transmit and receive ultrasound and generates ultrasound images based on output signals from the probe 101; a display 108 that displays the ultrasound images on a screen; and an operation input unit 110 that receives operation input from a user (operator). The probe 101 is connectable to the ultrasound diagnostic device 100 by a cable 102. Note that the probe 101 may be included as a function of the ultrasound diagnostic device 100, and the display 108 need not be included in the structure of the ultrasound diagnostic device 100.

### <Structure of Ultrasound Diagnostic Device 100>

The ultrasound diagnostic device 100 includes a transmitter 103, a receiver 104, an ultrasound imaging signal generator 105, an imaging signal synthesizer 106, a digital scan converter (DSC) 107, the display 108, and a controller 109. The transmitter 103 selects each transducer used when transmitting or receiving, from among the transducers 101a of the probe 101, and controls the timing of application of a high voltage to each of the transducers 101a of the probe 101 to cause transmission of ultrasound waves via a multiplexer (not illustrated) that secures input and output for the transducers selected. The receiver 104 amplifies, performs analog to digital (A/D) conversion, and performs reception beamforming on electric signals obtained from the transducers 101a based on reflected ultrasound waves received by the probe 101, to generate acoustic line signals (delay and sum (DAS) data). The ultrasound imaging signal generator 105 has a harmonic component extractor 105a that extracts a harmonic component from an acoustic line signal, which is an output signal from the receiver 104, performs processing such as envelope detection and logarithmic compression on the acoustic line signal and the harmonic component to perform luminance conversion, and generates an ultrasound image (B-mode image) by subjecting a resulting luminance signal to coordinate conversion in a rectangular coordinate system. The imaging signal synthesizer 106 includes an image memory 106a and synthesizes ultrasound imaging signals by synthesizing ultrasound image sub-frame data and the like. The DSC 107 outputs ultrasound image frame data to the display 108. The controller 109 controls the components of the ultrasound diagnostic device 100. Further, the ultrasound diagnostic device 100 may include a data storage that stores acoustic line signals output by the receiver 104 and ultrasound image signals output by the ultrasound imaging signal generator 105.

Of these components, the transmitter 103, the receiver 104, the ultrasound imaging signal generator 105, and the imaging signal synthesizer 106 constitute an ultrasound signal processor 150. The ultrasound signal processor 150 includes ultrasound signal processing circuitry.

Components of the ultrasound diagnostic device 100, for example the transmitter 103, the receiver 104, the ultrasound imaging signal generator 105, the imaging signal synthesizer 106, the DSC 107, and the controller 109, may each be implemented by hardware circuitry such as a field programmable gate array (FPGA) or application specific integrated circuit (ASIC). Alternatively, components of the ultrasound diagnostic device 100 may be implemented by a programmable device such as a central processing unit (CPU), general-purpose computing on a graphics processing unit (GPU), a processor, or the like, and software. These components can each be a single circuit component or an assembly of circuit components. Further, a plurality of components can be combined into a single circuit component or can be an aggregate of a plurality of circuit components.

The image memory 106a and the data storage are each a computer-readable storage medium, and may be a flexible disk, a hard disk, magneto-optical (MO), a digital versatile disc (DVD), digital versatile disc random access memory (DVD-RAM), semiconductor memory, or the like. Further, the image memory 106a and the data storage may be a storage device externally connected to the ultrasound diagnostic device 100.

The ultrasound diagnostic device 100 pertaining to Embodiment 1 is not limited to the structure illustrated in FIG. 2. For example, a given structure might not require a particular component, or the probe 101 may incorporate the transmitter 103, the receiver 104, or a portion of either or both of the transmitter 103 and the receiver 104.

The ultrasound diagnostic device 100 pertaining to Embodiment 1 is characterized by the ultrasound signal processor 150 that comprises the transmitter 103, the receiver 104, the ultrasound imaging signal generator 105, and the imaging signal synthesizer 106. Therefore, the present specification is mainly concerned with describing the structure and functions of each component of the ultrasound signal processor 150, and other structure may be the same as that used in known ultrasound diagnostic devices, and the ultrasound signal processor 150 pertaining to Embodiment 1 can be used in a known ultrasound diagnostic device.

The following is an overview of the probe 101 externally connected to the ultrasound diagnostic device 100 and of structure other than the ultrasound signal processor 150 of the ultrasound diagnostic device 100.

The probe 101 includes the transducers 101a arrayed in, for example, a one-dimensional direction (also referred to as an “azimuth direction”). The probe 101 converts a pulsed electric drive signal supplied from the transmitter 103 (also referred to as a “drive pulse signal”) into pulsed ultrasound. The probe 101 transmits an ultrasound beam composed of a plurality of ultrasound waves emitted from a plurality of transducers towards a measurement object while a transducer-side outer surface of the probe 101 is in contact with a skin surface of a subject. The probe 101 receives a plurality of ultrasonic reflected waves from the subject (also referred to as “reflected waves”), converts the reflected waves into electric signals via a plurality of transducers, and supplies the electric signals to the receiver 104. According to Embodiment 1, the probe 101 includes 192 elongate transducers 101a. The transducers 101a may be arranged in a two-dimensional array.

The operation input unit 110 receives various operation inputs such as settings and operations with respect to the ultrasound diagnostic device 100 from a user for inputting, for example, a command to start diagnosis or data such as personal information about the subject, and outputs to the controller 109. The operation input unit 110 may be, for example, a touch panel integrated with the display 108. In such a case, various settings and operations of the ultrasound diagnostic device 100 can be performed by touch or drag operations with respect to operation keys displayed on the display 108, and the ultrasound diagnostic device 100 is configured to be operable via the touch panel. Further, the operation input unit 110 may be, for example, a keyboard with keys for various operations, or an operation panel with buttons, levers, and the like for various operations. Further, a trackball, a mouse, a flat pad, or the like for moving a cursor on the display 108 may be included. Further, a plurality of these input options may be used, or a combination of these input options may be used.

The display 108 is a display device for image display and displays on a screen an image output from the DSC 107. The display 108 may include a liquid crystal display (LCD), a cathode-ray tube (CRT), an organic electroluminescence (EL) display, or the like.

### <Structure of Ultrasound Signal Processor 150>

The following describes the transmitter 103, the receiver 104, the ultrasound imaging signal generator 105, and the imaging signal synthesizer 106 that constitute the ultrasound signal processor 150.

### (Transmitter 103)

The transmitter 103 is connected to the probe 101 via the cable 102, and is circuitry that controls timing of high voltage application to each transducer included in an array of transmission transducers that may be all or a portion of the transducers 101a of the probe 101, in order to cause transmission of ultrasound from the probe 101. The transmitter 103 selects an array of transmission transducers from the transducers 101a of the probe 101 to supply a drive signal to, and causes transmission of an ultrasound beam focused on a transmission focal point from the transmission transducers. At this time the transmitter 103 generates a drive pulse signal including, for example, three frequencies of fundamental wave f1, f2, f3 components as a drive signal, such that drive pulse signals having different frequency distributions can be applied to an array of transmission transducers. In the present description, a unit of transmission in which an ultrasound beam is transmitted and reflected waves are received is referred to as a “transmission event”.

In the ultrasound diagnostic device 100, the transmitter 103 selects an array Txq (q=1 to qmax, where q is a natural number) of transmission transducers from the transducers 101a, and causes transmission of an ultrasound beam focused on a transmission focal point FP from the array Txq of the transmission transducers.

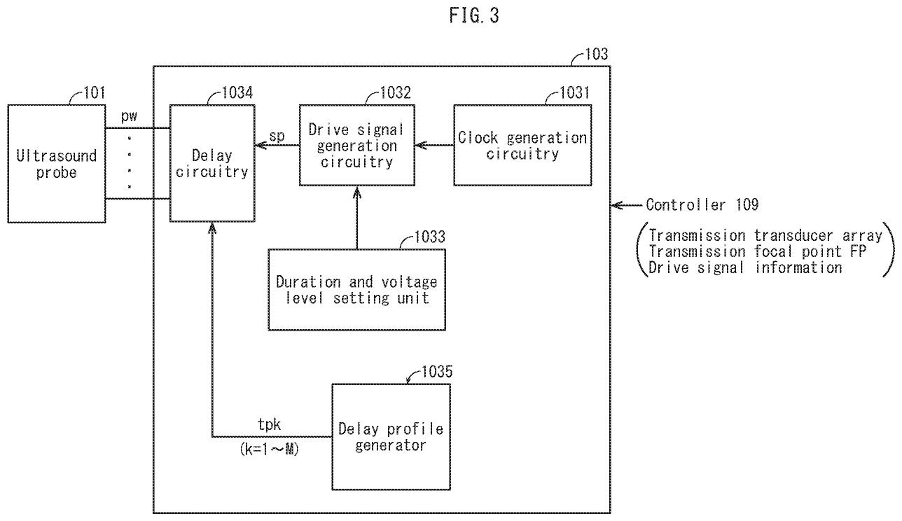

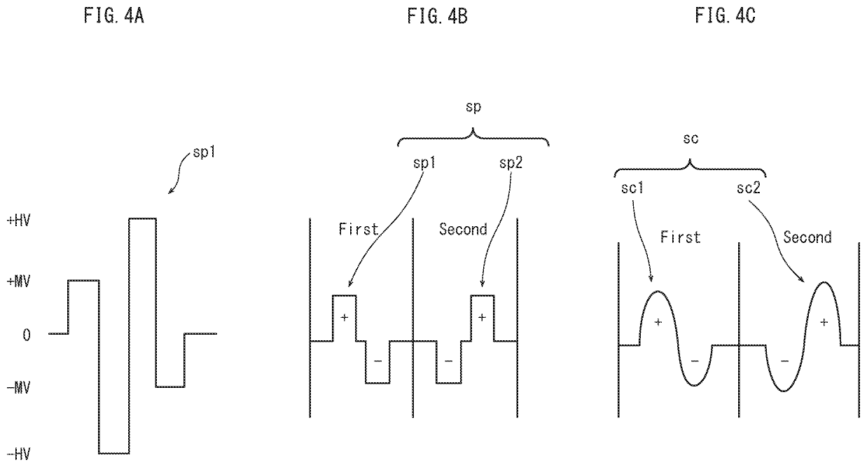

FIG. 3 is a function block diagram illustrating structure of the transmitter 103. As illustrated in FIG. 3, the transmitter 103 includes clock generation circuitry 1031, drive pulse signal generation circuitry 1032, a duration and voltage level setting unit 1033, delay circuitry 1034, and a delay profile generator 1035. FIG. 4A and FIG. 4B are schematic diagrams illustrating an example sp of a drive pulse signal generated in the transmitter 103 and an aspect of phase inversion transmission in pulse inversion. FIG. 4C is a diagram illustrating an example sc of a drive pulse signal generated by another method. A drive pulse signal whose voltage level changes steplessly, such as the drive pulse signal sc, may be obtained by a method of generating a drive pulse signal of arbitrary shape using a linear amplifier, or by a method of smoothing by band limiting processing or the like applied to the drive pulse signal sp and outputting a result as the drive pulse signal sc. As described above, a method using a rectilinear signal as a drive pulse signal or a method using a drive pulse signal that changes steplessly like the drive pulse signal sc can be selected according to requirements.

### [Clock Generation Circuitry 1031]

The clock generation circuitry 1031 is circuitry that generates a clock signal that is a minimum unit of time of output timing control of the drive pulse signal sp and duration control of each voltage level.

### [Drive Pulse Signal Generation Circuitry 1032, Duration and Voltage Level Setting Unit 1033]

The drive pulse signal generation circuitry 1032 generates a drive pulse signal sp for causing transmission of an ultrasound beam from transducers included in the array Tx of transmission transducers, based on output from the duration and voltage level setting unit 1033.

The drive pulse signal generation circuitry 1032 causes the drive pulse signal sp to be generated with a rectilinear waveform by switching between and outputting, for example, a 5 value (+HV/+MV/0(GND)/−MV/−HV) voltage or 3 value (+HV/0(GND)/−HV) voltage, as illustrated in FIG. 4A. Absolute values of amplitude of the drive pulse signal, positive and negative voltage identities, and the number of voltage steps are not limited to the examples described above.

Further, the ultrasound diagnostic device 100 can use a pulse inversion method, for example, to extract harmonic components in tissue harmonic imaging (THI). When the pulse inversion method is performed, the drive pulse signal generation circuitry 1032 generates a pair of continuous drive pulse signals sp1 and sp2 having inverted phases. As a result, as illustrated in FIG. 4B, a first drive pulse signal sp1 and a second drive pulse signal sp2 generated by the drive pulse signal generation circuitry 1032 have inverted phases.

If required, a configuration may be used in which the first drive pulse signal sp1 and the second drive pulse signal sp2 do not have a symmetrical shape of inverted phases, and a portion thereof may be asymmetrical with a linear signal component.

Further, methods of extracting harmonics are not limited to use of phase inversion, and a known amplitude modulation method may be used, for example.

In addition, as a method of calculating reception results of a plurality of transmission events and extracting a required reception signal component, the number of transmission events is not limited to two, and may be three or more. For example, reception results of three transmission events in which the phase of the drive pulse signal is shifted by 120° each event may be combined to extract a third harmonic component.

### [Delay Profile Generator 1035]

The delay profile generator 1035 is circuitry that sets delay times tpk (where k is a natural number from 1 to M, where M is the number of transducers included in the array Tx of transmission transducers) that determine transmission timing of an ultrasound beam for each transducer, based on information from a transmission control signal from the controller 109 indicating positions of the array Tx of transmission transducers and the transmission focal point FP, and outputs the delay times to the delay circuitry 1034. Thus, transmission of an ultrasound beam is delayed for each transducer by the corresponding delay time to achieve electron focusing of the ultrasound beam.

### [Delay Circuitry 1034]

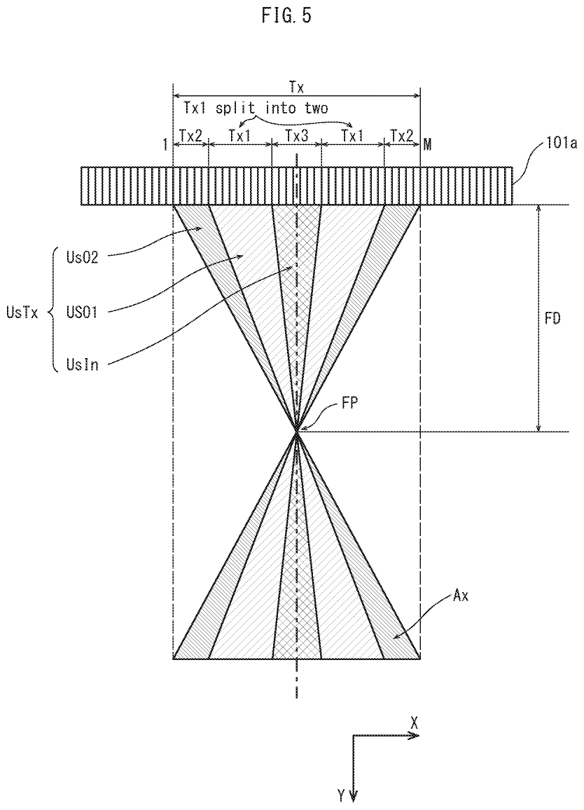

The delay circuitry 1034 is circuitry that sets delay times for each transducer based on a delay profile for transmission timing of a transmission pulse, such that drive signal transmission is delayed by the set delay time in order to focus an ultrasound beam. More specifically, based on the drive pulse signal sp from the drive pulse signal generation circuitry 1032 and the delay times tpk from the delay profile generator 1035, the delay circuitry 1034 performs transmission processing that supplies a drive signal pw for causing transducers included in the array Tx of transmission transducers among the transducers 101a of the probe 101 to transmit an ultrasound beam. When transmitting “forwards” from the probe 101, in the drive signal pw, a large delay time tpk is applied to transducers positioned centrally in the array Tx of transmission transducers. As a result, as illustrated in FIG. 5, a focused ultrasound beam is transmitted from the array Tx of transmission transducers to a specific site in a subject corresponding to a transmission focal point FP.

### [Transmitted Ultrasound Beam]

FIG. 5 is a schematic diagram illustrating an example of a propagation path of an ultrasound beam pertaining to transmission according to the transmitter 103 when “partial transducer array transmission” is executed according to the present disclosure. A row of transducers arranged in an array that contributes to ultrasound transmission is illustrated as the array Tx of transmission transducers. As illustrated in FIG. 5, in this description an array direction (azimuth direction) of the transducers 101a is defined as an X direction, and a depth direction of a subject perpendicular to the azimuth direction is defined as a Y direction.

According to the transmitter 103, transmission timing of each transducer is set such that transmission timing is delayed most for transducers positioned at a center of the array Tx of transmission transducers selected from the transducers 101a, and therefore a wavefront of ultrasound transmitted from transducers in the array Tx of transmission transducers ideally converges at a focal point FP at a defined point at a certain depth in a subject. A focal depth FD of the transmission focal point FP can be set arbitrarily based on the delay profile described above. A wavefront converging at the transmission focal point FP diffuses again and an ultrasound transmission wave propagates in an hourglass-shaped space bounded by two straight lines intersecting at the transmission focal point FP with the array Tx of transmission transducers as a base. An area of this hourglass-shape (indicated by hatching in the drawing) is referred to as an ultrasound irradiation area Ax.

Note that in the present disclosure, converging of an ultrasound beam according to a transmitted wave means that an area irradiated by the ultrasound beam decreases after transmission to a minimum value at a specified depth, but the ultrasound beam is not limited to focusing on one point. When not focusing on one point, the “transmission focal point FP” indicates an ultrasound beam center at a depth at which the ultrasound beam converges.

Further, in the present disclosure, “partial transducer array transmission” means that a transducer array to be a transmission aperture is divided into partial transducer arrays for which frequency components, transmission drive voltage state transition timing, and the like are different, and is not to be confused with transmission apodization in which only transmission amplitude is changed.

The following describes a method of the ultrasound diagnostic device 100 of dividing the array Tx of transmission transducers into a plurality of transducer arrays and driving them accordingly.

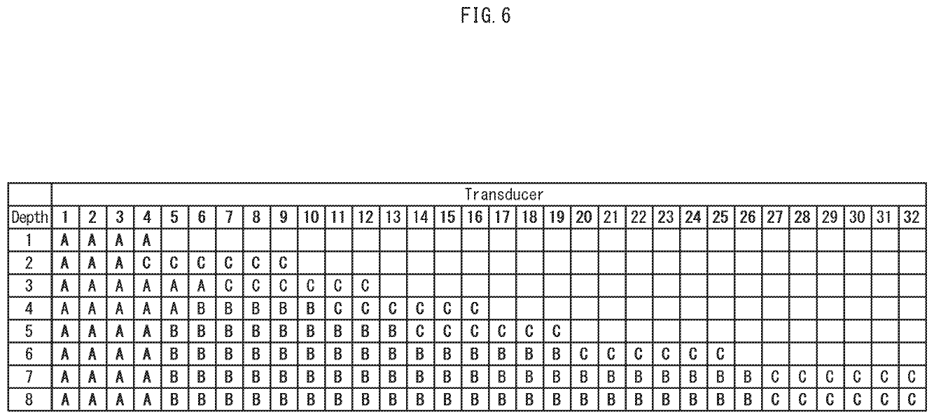

FIG. 6 is a diagram illustrating a relationship between focal depth FD of a transmission focal point FP and drive signal of a transmission transducer, with respect to an ultrasound beam transmitted according to the transmitter 103. In FIG. 6, the row direction indicates identification numbers of the transducers 101a, where “1” represents a first transducer from a center of the array Tx of transmission transducers and “32” represents a 32nd transducer. The column direction indicates identification numbers corresponding to positions of the transmission focal point FP in the depth direction divided into eight portions, where “1” represents a transmission focal point at a shallowest portion and “8” represents a transmission focal point at a deepest portion. Positions of “A, B, C” in FIG. 6 represent sections of transducers included in the array Txq (q=1 to qmax) of the transmission transducers, to which drive signals pwq (q=1 to qmax) are supplied independently. Further, “A, B, C” represent a division of the drive signals pw applied to transducers.

FIG. 6 illustrates only one side of a transmission aperture, and the side not illustrated is symmetrical to the one side with the center of the aperture as a line of symmetry. That is, at the depth “1”, a drive pulse signal “A” is supplied to eight transducers, or four transducers either side of a center of the transmission aperture, causing those eight transducers to transmit, and at the depth “8”, the drive pulse signal “A” is supplied to eight transducers around the center of the transmission aperture, a drive pulse signal “C” is supplied to six transducers at either end of the transmission aperture for a total of 12 transducers, and a drive pulse signal “B” is supplied to 44 transducers between the center and either end of the transmission aperture, in total causing 64 transducers to transmit. In the example illustrated in FIG. 6, the number of transducers is even, but transmission transducers may be divided into odd numbers.

That is, according to the ultrasound diagnostic device 100, as illustrated in FIG. 6, when the depth of the transmission focal point FP is “4” or more, the transmitter 103 selects the array Txq (where q=1 to qmax, q is a natural number, and qmax is 3 or greater) of transmission transducers from the transducers 101a, then supplies the drive signal pwq (q=1 to qmax) corresponding to “A, B, C” individually to each of the transmission transducers of the array Txq, causing transmission of an ultrasound beam focused on the transmission focal point FP from the array Txq of the transmission transducers, or “partial transducer array transmission”.

Thus, according to Embodiment 1, as illustrated in FIG. 5, when the transmission focal point FP is at depth “4” or greater, the transmitter 103 selects an array Tx3 of tertiary transducers that overlap in the azimuth direction with the transmission focal point FP, two arrays Tx1 of partial primary transducers that sandwich the array Tx3 in the azimuth direction, and two arrays Tx2 of secondary transducers that sandwich the arrays Tx1 in the azimuth direction, generates a drive signal having a different frequency distribution from that of the arrays Tx1 for the array Tx3 and the arrays Tx2, and supplies the drive signal. Here, the number of transducers in the array Tx3 may be from 1/16 to ½ of the transducers 101a in the array tx of transmission transducers. Likewise, the number of transducers in the arrays Tx2 may be from 1/16 to ½ of the transducers 101a in the array tx of transmission transducers.

Here, selection of transducers that make up the array Txq of transmission transducers is performed by the drive pulse signal generation circuitry based on an instruction from the controller 109. Further, assignment of the drive pulse signals sp to the array Txq of transmission transducers, and setting of duration for each section of the same voltage level and the voltage level itself for the drive pulse signals sp assigned to the array Txq are performed by the duration and voltage level setting unit 1033 based on an instruction from the controller 109, and applying the drive pulse signals sp to the array Txq is performed by the drive pulse signal generation circuitry 1032. Further, with respect to the drive pulse signals sp of the array Txq, durations and voltage levels of each section set by the duration and voltage level setting unit 1033 can be selectable according to input to the operation input unit 110, for example.

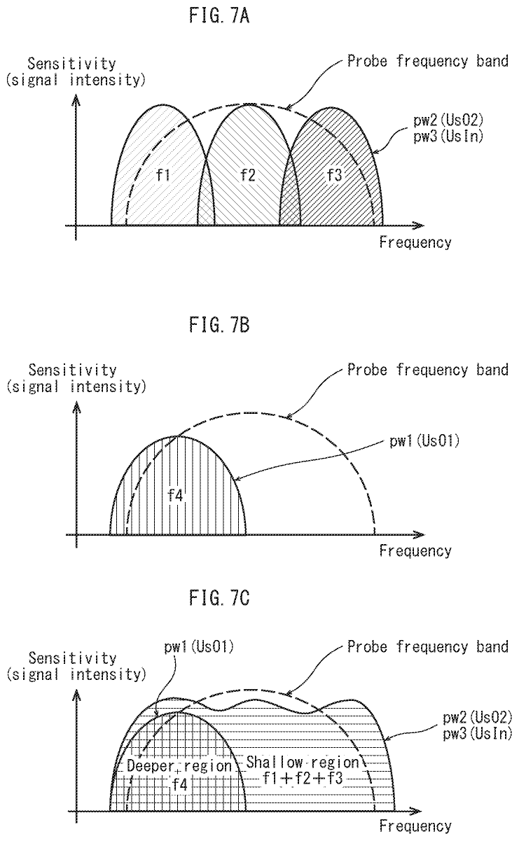

FIG. 7A, 7B, 7C are schematic diagrams illustrating examples of frequency distributions of ultrasound beams pertaining to transmission caused by the transmitter 103. When the ultrasound beams transmitted from the array Tx3, the arrays Tx1, and the arrays Tx2 are labelled UsIn, UsO1, and UsO2, respectively, FIG. 7A illustrates frequency distribution of UsIn and UsO2, FIG. 7B illustrates frequency distribution of UsO1, and FIG. 7C illustrates superimposed frequency distribution of UsIn, UsO1, and UsO2. In FIG. 7A, 7B, 7C, the horizontal axis represents frequency, the vertical axis represents signal intensity of a transmitted ultrasound pulse signal transmitted from transducers due to application of the drive signal pw, and the dashed line represents the transmission frequency band of the probe 101.

As illustrated in FIG. 5 and FIG. 7A, 7B, the transmitter 103 supplies different drive signals pw1, pw2, pw3 to the array Tx3, the arrays Tx1, Tx2, respectively, causing transmission of the ultrasound beam UsIn from the array Tx3, the ultrasound beam UsO1 from the arrays Tx1, and the ultrasound beam UsO2 from the arrays Tx2. As illustrated in FIG. 7A, pw2=pw3, or in other words in the drive pulse signal sections illustrated in FIG. 6, “A”=“C”, and at this time the ultrasound beams UsIn, UsO1, UsO2 are electron focused to converge on the same transmission focal point FP.

As illustrated in FIG. 7A, the frequency distributions of the drive signals pw2, pw3 supplied to the array Tx3 and the arrays Tx2 includes frequency components of fundamental waves f1, f2, f3. The frequency distributions of the transmitted ultrasound pulse signals of the drive signals pw2, pw3 are frequency bands included in a −20 dB transmission frequency band of the transducers 101a, and have intensity peaks lower and higher than a center frequency of the −20 dB transmission frequency band, and it is preferable that an intensity in the frequency band between the intensity peaks is −20 dB or more, based on maximum values of the intensity peaks. By setting an intensity between peaks to be −20 dB or more, transmission can be executed without splitting a time waveform peak of a transmitted ultrasound pulse even when there are a plurality of frequency intensity peaks. Here, focusing width of the ultrasound beam is proportional to the reciprocal of the frequency. For example, when the fundamental wave f3 has a frequency three times that of the fundamental wave f1, the fundamental wave has a beam width of ⅓ due to focusing. That is, the beam is focused at three times the density of the fundamental wave f1, and therefore sound pressure easily increases in the irradiation area including the component of the fundamental wave f3 even when a short axis of the ultrasound beam is focused by an acoustic lens or the like, and the beam reaches a non-linear region generating harmonics at a depth shallower than the electron focal point. This makes is possible to obtain a harmonic signal with a good signal to noise ratio from a shallow depth region.

On the other hand, the fundamental wave f1, which is a low frequency, can generate a high sound pressure area in a deeper region centered on the transmission focal point because a transmitted ultrasound wave has low attenuation in the shallow region and high reach in the deeper region. This contributes to obtaining a harmonic signal with a good signal to noise ratio.

With this frequency configuration it is possible to obtain a harmonic signal with a good signal to noise ratio from the shallow region to the deeper region in a transmission area of pw2 and pw3.

Accordingly, when compared to a case in which the drive signal pw3 is supplied only to the array Tx3, where a good signal is not obtained in the vicinity where a reflected wave from a shallow region directly faces a transmission direction and the shallow region is a region in which anisotropic reflection sites are common, such as a specular reflection member (puncture needle) or a tendon, it also possible to effectively receive reflected waves from an angle added to the transmission direction, and therefore visibility of a specular reflection member such as a puncture needle can be improved.

Further, as illustrated in FIG. 7B, the frequency distribution of the drive signal pw1 supplied to the arrays Tx1 include a frequency component of a low frequency band fundamental wave f4. Frequency distribution of the transmission pulse signal of the drive signal pw1 is a frequency band included in the −20 dB transmission frequency band of the ultrasound probe, and has a maximum intensity peak lower than the center frequency of the −20 dB transmission frequency band. Thus, the fundamental wave f4 has an effect of increasing azimuthal resolution and increasing speckle granularity in a deeper region, because the transmitted ultrasound has low attenuation in the shallow region and high reach in the deeper region. Further, the drive signal pw of the fundamental wave f4 does not include a high frequency component, and therefore contributes to a decrease in surface temperature of the probe 101. Further, sound pressure in a shallow region is not increased and unnecessary harmonics are not generated, and this contributes to an improvement in low echo extraction performance in the shallow region.

According to the ultrasound diagnostic device 100, the ultrasound beam UsO1 that has a smaller signal intensity in a high frequency band than the transmitted ultrasound beams UsIn, UsO2 is transmitted from the arrays Tx1, which reduces high frequency generation to suppress acoustic noise in a non-reception area in the ultrasound irradiation area Ax. Further, the ultrasound beam UsO1 that includes signal intensity in a low frequency band with little attenuation is transmitted from the arrays Tx1, improving depth of penetration such that applied energy can be used efficiently.

As described above, in the frequency distribution obtained by superimposing frequency distributions of UsIn, UsO1, UsO2, as illustrated in FIG. 7C, the −20 dB frequency band of the drive signal pw2 is wider from the −20 dB frequency band of the drive signal pw1. Further, a configuration can be adopted in which an ultrasound beam having a higher signal intensity in a high frequency band than the arrays Tx1 can be transmitted from the array Tx3 and the arrays Tx2.

Further, the relationship between frequency band signal properties of pw1 and pw3 is preferably such that, in the −20 dB transmission frequency band of the probe 101, pw3 substantially includes pw1, as illustrated in FIG. 7C. As a result, signal intensity being stronger in the UsO1 area than in the UsIn area at an aperture center in a deeper region beyond the focal point, such that a cross-section profile of the ultrasound beam is split into two peaks with a low center, can be avoided, as would happen if pw1 low frequency strength were higher than pw3 strength. Here, “substantially includes” means that each frequency component intensity of pw1 does not exceed that of pw3 by more than 6 dB within the −20 dB transmission frequency band of the probe 101.

FIG. 7C illustrates a preferred example in which pw2 and pw3 frequency bands have a plurality of signal intensity peaks, but having a plurality of signal intensity peaks is not an essential feature, and it suffices to have a frequency band signal higher than the frequency of pw1. For example, the drive signal may be for a broadband single signal intensity peak, and the signal intensity peak frequency is not limited to any particular example. However, even in this case it is preferable that the frequency band signal property of pw1 is included in pw3.

### (Receiver 104)

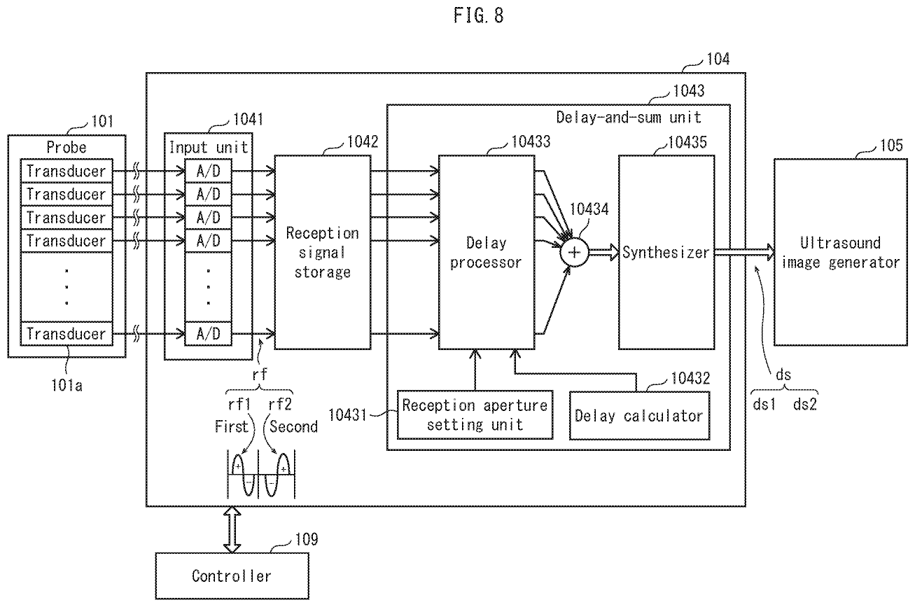

The receiver 104 generates acoustic line signals from electrical signals obtained by the transducers 101a, based on reflected ultrasound received by the probe 101. Here, an “acoustic line signal” is a reception signal for an observation point after delay-and-sum processing. Delay-and-sum processing is described in more detail later. FIG. 8 is a function block diagram illustrating structure of the receiver 104. As illustrated in FIG. 8, the receiver 104 includes an input unit 1041, a reception signal storage 1042, and a delay-and-sum unit 1043.

The following describes components of the receiver 104.

### [Input Unit 1041]

The input unit 1041 is circuitry connected to the probe 101 via the cable 102, and amplifies an electrical signal obtained by receiving an ultrasound reflected wave at the probe 101 in a transmission event, then generates an A/D converted reception signal (radio frequency (RF) signal). Reception signals are generated in a time series in an order of transmission events and output to the reception signal storage 1042, which stores the reception signals.

Here, a reception signal (RF signal) is a digital signal obtained by A/D conversion of an electrical signal converted from reflected ultrasound received by a transducer, and is composed of a series of signals that are continuous in a transmission direction (depth direction of subject) of ultrasound received by the transducer.

When pulse inversion is performed, the input unit 1041 receives a pair of phase-inverted rf signals rf1, rf2 based on reflected waves from a pair of polarity-inverted drive pulse signals sp1, sp2 or sc1, sc2 transmitted at time intervals on the same scan line.

The input unit 1041 generates a sequence of reception signals for a transmission event for each of the reception transducers Rw, based on reflected ultrasound obtained by the reception transducers Rw, where the reception transducers Rw are some or all of the N transducers 101a of the probe 101. The reception transducers Rw are selected based on an instruction from the controller 109. According to Embodiment 1, the reception transducers Rw are all N of the transducers 101a of the probe 101. Further, an array center of an array Rwx of the reception transducers Rw is selected so as to match an array center of the array Tx of transmission transducers, and the number of the reception transducers Rw may be equal to or greater than the number of transmission transducers.

### [Reception Signal Storage 1042]

The reception signal storage 1042 is a computer-readable storage medium, for example a semiconductor memory. The reception signal storage 1042 inputs a sequence of reception signals for each transmission event for each reception transducer from the transmitter 103, and may hold this data until a single ultrasound image is generated. Further, the reception signal storage 1042 may be a hard disk, magneto-optical (MO), digital versatile disc (DVD), digital versatile disc random access memory (DVD-RAM), or the like. Further, the reception signal storage 1042 may be a storage device that is external and connectable to the ultrasound diagnostic device 100. Further, the reception signal storage 1042 may be a part of the data storage.

### [Delay-and-Sum Unit 1043]

The delay-and-sum unit 1043 is circuitry that performs delay-and-sum processing of reception signal sequences received by each reception transducer from observation points in a calculation target area Bx in a subject for a transmission event, in order to generate acoustic line signals. Here, the “calculation target area Bx” is a unit of area for which acoustic line signal sub-frame data is generated according to delay-and-sum processing.

The delay-and-sum unit 1043 sets a plurality of calculation target areas BxI0 (where I0=1 to Imax; I0 is a natural number; and Imax is equal to 2 or more), each occupying a different position, and performs delay-and-sum processing for each observation point Pij in each of the calculation target areas BxI0, in a different position for each sub-frame, to generate acoustic line signal sub-frame data dsI0.

As illustrated in FIG. 8, the delay-and-sum unit 1043 includes a reception aperture setting unit 10431, a delay time calculator 10432, an addition unit 10434, and a synthesizer 10435. The following describes these components.

### i) Reception Aperture Setting Unit 10431

The reception aperture setting unit 10431 is circuitry that sets the calculation target area Bx corresponding to an analysis target range in a subject, and sets a reception aperture Rx based on positions of observation points Pij in the calculation target area Bx for which acoustic line signals are calculated. Here, the reception aperture Rx is an array of transducers selected from an array of reception transducers that received a reception signal, and when delay-and-sum processing is applied to reception signal sequences based on a reflected wave from observation points, the reception aperture Rx is an array of transducers that received reception signals that are a target of calculation. Further, according to the present description, when an observation point P is described by indices i and j that correspond to coordinates in the X direction and the Y direction, Pij is used to describe the observation point. In the delay-and-sum processing, delay times of reflected wave arrival from an observation point Pij to each reception transducer in a reception aperture Rx are calculated, and an acoustic line signal is calculated based on the delay times calculated for the observation point Pij.

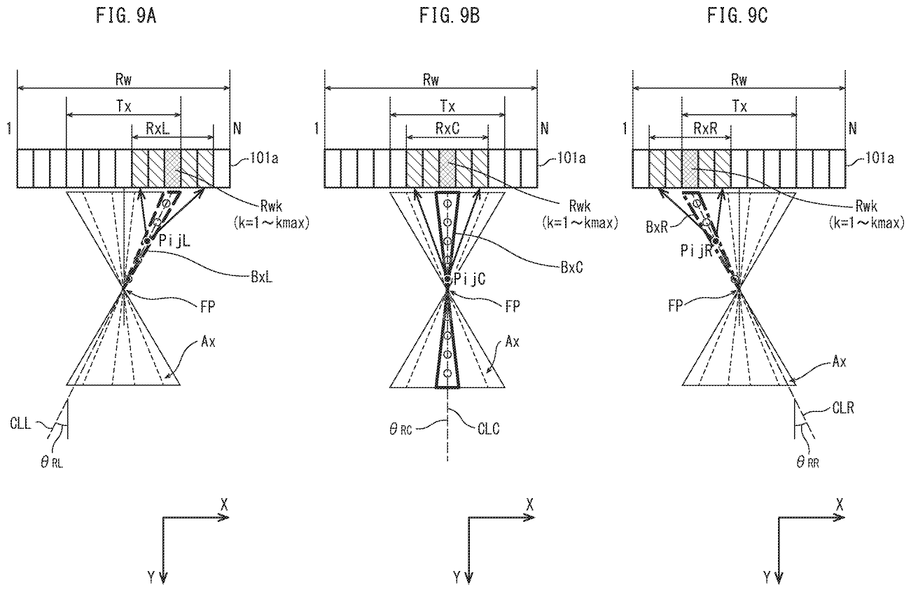

FIG. 9A, 9B, 9C are schematic diagrams for explanation of acoustic line signal generation for observation points Pij by the delay-and-sum unit 1043.

As illustrated in FIG. 9A, 9B, 9C, according to the ultrasound diagnostic device 100, the reception aperture setting unit 10431 sets calculation target areas BxL (Bx1), BxC (Bx2), BxR (Bx3) that each occupy a different position in the azimuth direction of the calculation target areas Bx, and performs delay-and-sum processing for observation points PijL, PijC, PijR in positions in the calculation target areas BxI0 (where I0=1 to 3) to generate acoustic line signal sub-frame data dsI0.

Here, the calculation target area Bx and the reception aperture Rx are set as follows.

The calculation target area BxC is set so that an observation point Pij is located in an area between two straight lines intersecting both ends of the array Tx3 and the transmission focal point FP. More specifically, as illustrated in FIG. 9B, the calculation target area BxC starts from an approximate center of the array Tx3 and is set in an area delineated by straight lines intersecting the transmission focal point FP (area corresponding to ultrasound beam UsIn in FIG. 5).

The calculation target areas BxL, BxR are set so that observation points Pij are located in areas between two straight lines intersecting both ends of the arrays Tx2 and the transmission focal point FP. More specifically, as illustrated in FIG. 9A, the calculation target area BxL starts from an approximate center of the array Tx2 to the right side of the drawing and is set in an area delineated by straight lines intersecting the transmission focal point FP (area corresponding to the right-side ultrasound beam UsO2 in FIG. 5). Similarly, as illustrated in FIG. 9C, the calculation target area BxR starts from an approximate center of the array Tx2 to the left side of the drawing and is set in an area delineated by straight lines intersecting the transmission focal point FP (area corresponding to the left-side ultrasound beam UsO2 in FIG. 5).

Further, the transducer array of the reception aperture RxC is set for delay-and-sum processing of observation points Pij located in the calculation target area RxC. More specifically, the reception aperture RxC is set such that an array center is positioned within the array Tx3, as illustrated in FIG. 9B.

The transducer arrays of the reception apertures RxL, RxR are set for delay-and-sum processing of observation points Pij location in the calculation target areas RxL, RxR, respectively. More specifically, the reception aperture RxL is set such that an array center is positioned with the array Tx2 to the right of the drawing as illustrated in FIG. 9A. More specifically, the reception aperture RxR is set such that an array center is positioned within the array Tx2 to the left of the drawing, as illustrated in FIG. 9C.

Further, for example, a configuration may be adopted in which an acoustic line signal is generated such that an approximate center of the array Tx3 or the arrays Tx2 are fixed as a center of the reception aperture Rx.

The center of the reception aperture R according to the present disclosure does not mean a physical center of the reception transducer array, but refers to a start point of a reception acoustic line, or in other words a reference point in reception delay calculation.

Assuming that a direction perpendicular to the azimuth direction is the depth direction and straight lines intersecting centers in the azimuth direction of the calculation target areas BxL, BxC, BxR are scanning lines CLL, CLC, CLR, as illustrated in FIG. 9A, 9B, 9C, angles θ of the scanning lines CLL, CLC, CLR with respect to the depth direction are reception steering angles θRL, θRC, θRR.

According to the present embodiment, a calculation target area Bx with a large reception steering angle θR may be configured to be shorter in the depth direction than a calculation target area Bx with a small angle.

More specifically, as illustrated in FIG. 9A, 9B, 9C, the reception steering angles θRL, θRR with respect to the depth direction Y of the area center lines CLL, CLR are larger than the reception steering angle θRC with respect to the depth direction Y of the area center line CLC. Thus, the calculation target areas BxL, BxR can be shallower in the depth direction than the calculation target region BxC. In the example illustrated in FIG. 9A, 9B, 9C, the transmission steering angle is 0°, and therefore among the calculation target areas BxL, BxC, BxR, the calculation target areas having a large angle between the transmission steering angle and the area center lines CLL, CLR can be set shorter in the depth direction than a calculation target area having a small angle.

For example, anisotropic high-reflection members such as a high-angle puncture needle shaft, a longitudinal boundary, an anterior talofibular ligament, and the like tend to be located in a shallow region or peripheral region thereof. Thus, in order to receive reflected waves from such anisotropic high-reflection members, it is effective to increase the reception steering angle θR in the shallow region.

However, even if a calculation target area Bx with a large reception steering angle θR is enlarged to a deeper region, an overlapping width between the calculation target areas BxL, BxC, BxR in the deeper region is small or there is no overlap. Thus, there is no function of enhancing image rendering by superimposing images as a spatial compound.

Further, even if a calculation target area Bx is expanded to a deeper region, a transmission path for transmission and reception is long and attenuation is large, and transmission and reception sensitivity of transducers also decreases as the angle increases, so that it can be difficult to obtain sufficient spatial resolution and signal to noise ratio in a generated image.

On the other hand, among the calculation target areas BxL, BxC, BxR, setting the calculation target areas BxL, BxR to be shorter in the depth direction than the calculation target area BxC does not have the demerits described above, a viewing angle is effectively expanded by efficient use of resources for calculation, and reflected waves from an anisotropic high-reflection member in a shallow region are efficiently received, increasing visibility.

According to Embodiment 1, the calculation target area Bx for which acoustic line signal sub-frame data is generated starts from an approximate center of the transmission aperture array Tx3 or the arrays Tx2 and is set within an area delineated by straight lines that intersect the transmission focal point FP (an area corresponding to the ultrasound beams UsIn, UsO2 in FIG. 5), as described above. That is, according to the transmission example in FIG. 5, the calculation target area BxC has a reception steering angle of 0°, and the calculation target areas BxL, BxR have steering angles equal to the angle between the Y direction and lines from approximate center positions of the arrays Tx2 towards the transmission focal point FP, thereby determining sub-frame reception steering angles θRL, θRR. However, the calculation target areas Bx are not limited to these examples, and may be set any arbitrary area included in an area corresponding to the ultrasound beams UsIn and UsO2 in FIG. 5.

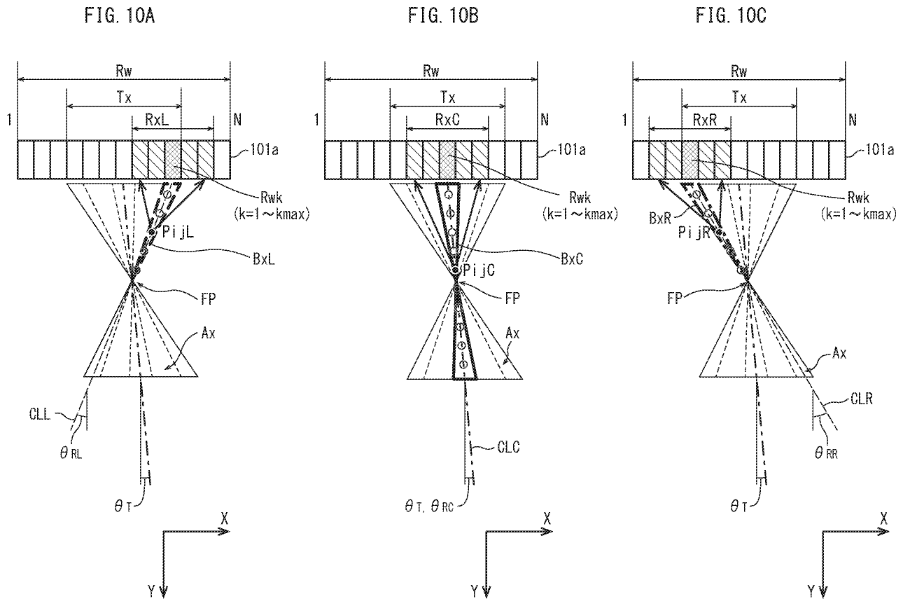

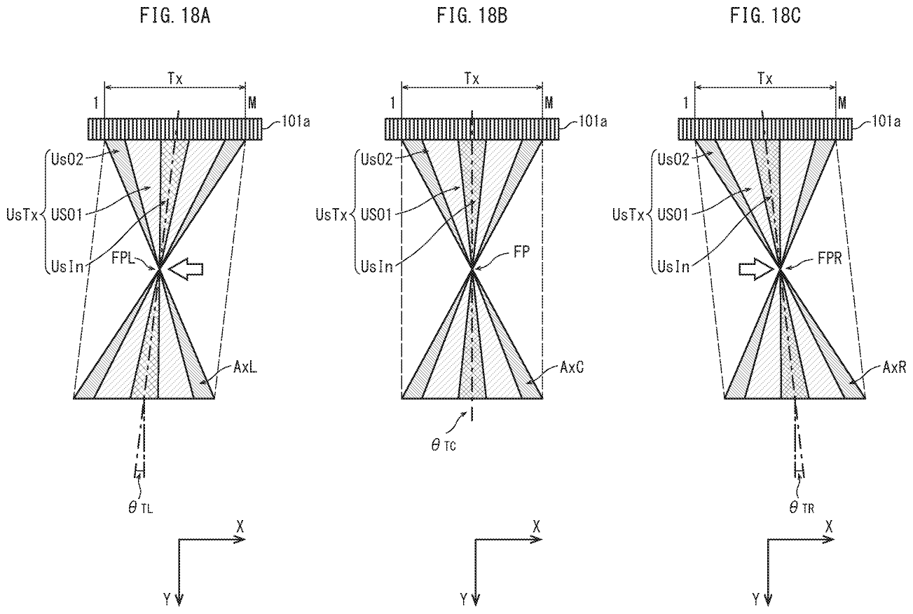

FIG. 10A, 10B, 10C are schematic diagrams for explanation of acoustic line signal generation for observation points PijL, PijC, PijR by the delay-and-sum unit 1043 when a transmission steering angle θT is added. Modification 1, in which transmission is performed with a transmission steering angle to be described later, is an aspect of the calculation target area Bx illustrated in FIG. 18A, 18C. According to Modification 1, as in FIG. 9A, 9C, the calculation target area Bx is set in an area of UsIn, UsO2.

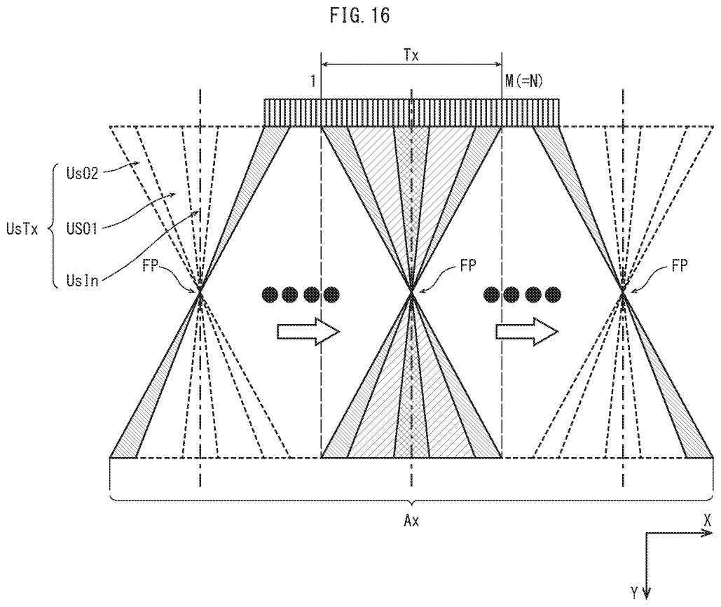

If transmission sub-scanning is terminated when a transmission aperture center reaches a last transducer, as would normally be the case, the calculation target areas BxL, BxR would not reach a last transducer, causing image loss at ends of the probe, and therefore, as illustrated in FIG. 16, it is preferable to perform transmission sub-scanning while virtually increasing the number of probe transducers until the calculation target areas BxL, BxR reach ends of the probe.

Further, one aspect of setting the reception aperture Rx for the observation points PijL, PijC, PijR of the areas BxL, BxC, BxR by the reception aperture setting unit 10431 is a method of fixing an approximate center of the array Tx3 or the arrays Tx2 as a center of the reception aperture Rx to generate acoustic line signals. With progress of a transmission wavefront, the aperture center does not move as the wavefront moves past observation points Pij in the transmission focal point direction, that is, a reception focal point position is moved according to transmission wavefront progression in the UsIn, UsO2 area for acoustic line signal generation. As a result, a progress direction of a transmitted wavefront and a reception direction are substantially the same, and a reflected wave can be received from a wide viewing angle according to BxL, BxR, where it would be difficult to efficiently receive with BxC alone. Thus, for example, visibility can be improved for a specular reflection member or an anisotropic site located in a shallow region or peripheral region thereof, such as a high-angle puncture needle, a longitudinal boundary between different tissues, an anterior talofibular ligament, or the like.

In the examples illustrated in FIG. 10A, 10B, 10C, among the calculation target areas BxL, BxC, BxR, the calculation target areas with a large angle between a direction indicated by a transmission steering angle θT and the area center lines CLL, CRR can be set shorter in the depth direction than that of a calculation target area with a small angle.

### ii) Delay Time Calculator 10432

The delay time calculator 10432 is circuitry that calculates delay times for reflected wave arrival at each reception transducer in a reception aperture Rx from an observation point Pij, with respect to each observation point Pij in a calculation target area Bx corresponding to an analysis target range in a subject.

A transmission wave radiated from the array Tx of transmission transducers reaches an observation point Pij, generates a reflected wave at the observation point Pij according to a change in acoustic impedance, and the reflected wave returns to a reception transducer Rw in a reception aperture Rx of the probe 101. A length of a path to any observation point Pij and lengths of paths from any observation point Pij to each reception transducer Rw can be calculated geometrically.

More specifically, calculation of delay times for an observation point Pij is performed as follows.

The delay time calculator 10432 calculates arrival time differences (delays) of reflected ultrasound to each reception transducer Rw by dividing differences in distances between an observation point Pij and each reception transducer Rw by a sound velocity value Cs, for each observation point Pij in a calculation target area Bx, based on sequences of reception signals at reception transducers Rw in a reception aperture Rx. More specifically, as illustrated in FIG. 9A, 9B, 9C, for each transmission event, the delay time calculator 10432 geometrically calculates lengths of paths from an observation point Pij to each reception transducer Rwk (where k=1 to kmax), based on information indicating positions of reception transducers Rw and information indicating a position of the observation point Pij. Then, differences Δdk in path length from the observation point Pij to each of the reception transducers Rwk are divided by the sound velocity value Cs, and delay times Δtk of reflected wave arrival at each of the reception transducers Rw from the observation point Pij are calculated for each of the reception transducers Rwk.

iii) Delay Processor 10433

The delay processor 10433 is circuitry that generates acoustic line signals ds for an observation point Pij using reference delay times for each reception transducer Rw.

First, a reception signal value for an observation point Pij is specified as follows.

The delay processor 10433 calculates arrival times of a reflected wave to each reception transducer Rw from the observation point Pij based on arrival time differences (delays) calculated by the delay time calculator 10432, and identifies reception signals corresponding to reception transducers Rw based on the arrival times. More specifically, the delay processor 10433 calculates an ultrasound round-trip time between the observation point Pij and a reception transducer Rw closest to the observation point Pij, and calculates reflected wave arrival times to the reception transducers Rw by adding the arrival time differences (delays) calculated by the delay time calculator 10432. The delay processor 10433 then reads a reception signal sequence RFk from the reception signal storage 1042, and specifies a reception signal value corresponding to an arrival time of a reflected wave for each reception transducer Rw. Thus, a reception signal value is specified for each of the reception transducers Rw. The delay processor 10433 performs this processing for all of the observation points Pij included in the calculation target area Bx, and calculates delay times Δtk and specifies reception signals for each of the reception transducers Rwk.

### iv) Addition Unit 10434

The addition unit 10434 is circuitry that receives, as input, reception signals specified as corresponding to reception transducers Rwk output from the delay processor 10433 and performs addition to generate delay-and-sum acoustic line signals with respect to the observation points Pij. The addition unit 10434 may be configured to multiply reception signals specified as corresponding to reception transducers Rwk by a weighting sequence (reception apodization) then perform addition to generation acoustic line signals with respect to the observation points Pij. In such a case, the weighting sequence preferably assigns transducers positioned centrally in the array direction of a reception aperture Rx the greatest weight, with a symmetrical distribution about the transmission focal point F. As a shape of a weighting sequence distribution, a Hamming window, a Hann window, a rectangular window, or the like can be used, and the shape of distribution is not limited to any particular example.

Due to the delay processor 10433 compensating for delay times of reception signals detected by the reception transducers Rw in the reception aperture Rx, and the addition unit 10434 performing addition processing, reception signals received by reception transducers Rw based on reflected waves from an observation point P are superimposed to increase the signal to noise ratio, such that the reception signals from the observation point P can be extracted.

The delay processor 10433 generates acoustic line signals for all observation points P in a calculation target area Bx. Positions of observation points Pij to be calculated are such that, for example, acoustic line signals are generated for all observation points Pij in a calculation target area Bx by repeating ultrasound transmission while gradually moving in a scanning line and azimuth direction, and gradually output to the synthesizer 10435.

### v) Synthesizer 10435

The synthesizer 10435 is circuitry that generates acoustic line signal sub-frame data from acoustic line signals of calculation target areas Bx. The synthesizer 10435 gradually inputs acoustic line signals from the addition unit 10434 generated for observation points Pij in a calculation target area Bx, and uses positions of observation points P from which acoustic line signals are acquired as an index to superimpose acoustic line signals with respect to observation points P to generate acoustic line signal sub-frame data.

As described above, the reception aperture setting unit 10431 sets a plurality of calculation target areas BXI0. Thus, for each of the calculation target areas BxI0, the delay time calculator 10432, the delay processor 10433, the addition unit 10434, and the synthesizer 10435 in order perform delay-and-sum processing for each observation point Pij in the calculation target area BxI0 such that the synthesizer 10435 generates acoustic line signal sub-frame data corresponding to the calculation target areas BxI0. Synthesized frame acoustic line signals are output in order to the ultrasound imaging signal generator 105.

### (Ultrasound Imaging Signal Generator 105)

The ultrasound imaging signal generator 105 converts acoustic line signal sub-frame data and the like corresponding to calculation target areas BxI0 into luminance signals corresponding to intensity, and converts the luminance signals into an orthogonal coordinate system to generate ultrasound imaging signal sub-frame data. The ultrasound imaging signal generator 105 sequentially performs this processing for each of the calculation target areas BxI0, and sequentially outputs generated ultrasound imaging signal sub-frame data to the imaging signal synthesizer 106. More specifically, the ultrasound imaging signal generator 105, after generation of a broadband acoustic line signal by extracting harmonic components using a pulse version method with respect to an acoustic line signal obtained from the delay-and-sum unit 1043, generates ultrasound imaging signal sub-frame data and the like by processing such as envelope detection and logarithmic compression to perform luminance conversion, and subjecting the luminance signal to coordinate conversion in an orthogonal coordinate system. That is, an ultrasound imaging signal may be a B-mode imaging signal in which intensity of ultrasound reception signals is represented by luminance

Further, according to the present disclosure, “ultrasound imaging signal” refers to a signal of each stage displayed as an image generated based on acoustic line signals, and includes not only luminance information that is a final stage for imaging but also a preceding stage of a reception signal after envelope detection, a reception signal after signal processing such as band-pass filter processing, and the like.

Further, the ultrasound imaging signal generator 105 includes a harmonic component extractor 105a, and generates an ultrasound imaging signal from harmonic components extracted by a pulse version method by using the harmonic component extractor 105a.

At this time, the harmonic component extractor 105a extracts a harmonic component by performing a pulse inversion method with respect to an acoustic line signal output from the receiver 104, as described in JP 2015-112261, for example. Among harmonic components, even-order harmonic components can be extracted when a fundamental wave component included in a reception signal is removed by addition processing of acoustic line signals based on pairs of phase-rotated if signals rf1, rf2, based on reflected waves corresponding to two transmission ultrasound waves generated by a pair of the drive pulse signals sp1, sp2, whose phases are inverted when transmitted at time intervals along the same scan line, as described above. Odd-order harmonic components can be extracted by subtraction processing of acoustic line signals based on the pair of rf signals rf1, rf2 to remove even-order harmonic components, then performing filter processing as necessary. Extracted even-order harmonic components and odd-order harmonic components are subjected to phase adjustment processing using an all-pass filter or the like, then addition processing to obtain a broadband acoustic line signal.

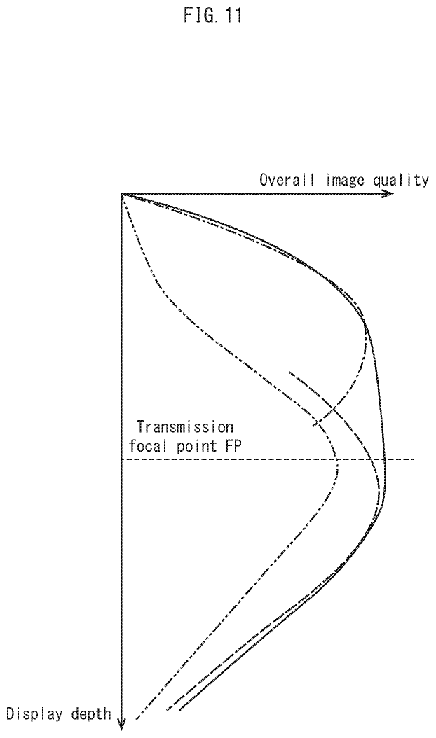

FIG. 11 illustrates a relationship between display depth and overall image quality in an ultrasound image generated by the ultrasound imaging signal generator 105 when a transmission method described in JP 2014-168555 or JP 2016-214622 is applied as transmission ultrasound of pw3 (UsIn), pw2 (UsO2) as in FIG. 7C. In FIG. 11, a line of alternating long and short dashes indicates a harmonic component generated by a fundamental wave f3 component, and a line of long dashes indicates a harmonic component generated by fundamental wave f1, f2 components. A solid line indicates a frequency property of overall image quality that combines both, and a line of alternating long and two short dashes indicates overall image quality according to a conventional technique. In contrast, according to FIG. 7C, pw2 (UsO1), only a low frequency component of transmitted sound is used, and therefore only the focusing by an acoustic lens in the shallow region does not sufficiently increase sound pressure and generation of a harmonic signal is very slight. As a result, in an area shallower than the focal point FP in FIG. 5, harmonics with a good signal to noise ratio are generated by the focusing by an acoustic lens in the area of UsIn, UsO2, and harmonics generation in the area of UsO1 is small. In this way, by setting the observation points Pij in the UsIn, UsO2 areas along with uneven spatial control of harmonics generation, it is possible to receive a harmonic signal with high signal to noise ratio, and to prevent scattering and reflection of acoustic noise from the UsO1 areas in which observation points are not set, in order to obtain images with both good reflector rendering due to the high signal to noise ratio and excellent no/low echo rendering due to suppression of acoustic noise contamination The pwl transmitted in the UsO1 area does not contribute to generation of harmonics in the shallow region, but does contribute to an increase in sound pressure in a region near to the focal point FP due to focusing by electron focus. According to the ultrasound diagnostic device 100, observation points Pij are set in UsIn, UsO2, which are high harmonic generation areas, so that an ultrasound image composed of a reception signal with high signal to noise ratio and small acoustic noise contamination can be obtained from three directions. Displaying an image after synthesis by an imaging signal synthesizer (described below) obtains an ultrasound image in which visibility of an anisotropic reflection target such as a specular reflection member like a puncture needle, or a tendon, or the like is improved, without lowering frame rate.

### (Imaging Signal Synthesizer 106)

The imaging signal synthesizer 106 is circuitry that synthesizes ultrasound imaging signal sub-frame data and the like corresponding to calculation target areas BxI0 output from the ultrasound imaging signal generator 105 with reference to positions of observation points to generate ultrasound image frame data and the like. Here, “frame” indicates a unit of one integrated signal necessary for forming one ultrasound image. One frame-worth of combined acoustic line signals may be referred to as “acoustic line signal frame data”.

The imaging signal synthesizer 106 includes an image memory 106a constituted by a semiconductor memory such as dynamic random-access memory (DRAM), static random-access memory (SRAM) included in an integrated circuit, or the like. The imaging signal synthesizer 106 stores ultrasound imaging signal sub-frame data and the like corresponding to calculation target areas Bx output from the ultrasound imaging signal generator 105.

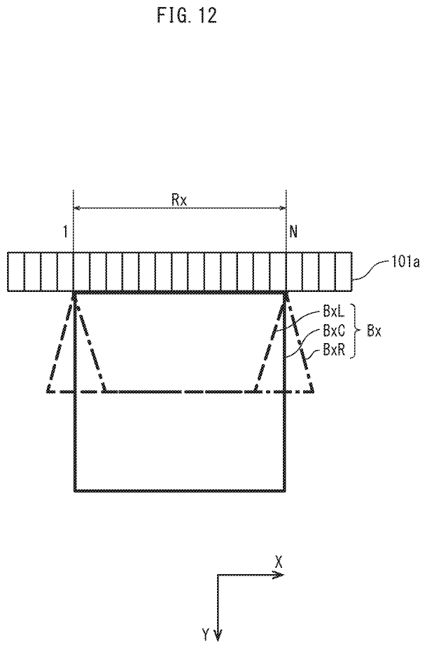

FIG. 12 is a schematic diagram for explaining an example of generation of ultrasound image frame data in the imaging signal synthesizer 106. As illustrated in FIG. 12, calculation target areas BxL, BxC, BxR having different positions in the azimuth direction and different ranges in the depth direction are set, delay-and-sum processing is performed for observation points Pij in each of the calculation target areas BxI0 to generate acoustic line signal sub-frame data dsI0, and the ultrasound imaging signal generator 105 generates ultrasound imaging signal sub-frame data corresponding to each of the calculation target areas BxI0.

When the imaging signal synthesizer 106 stores ultrasound imaging signal sub-frame data corresponding to calculation target areas Bx in the image memory 106a, an acoustic line signal calculated for an observation point Pij is stored at an address of the image memory 106a corresponding to a position of the observation point Pij in order to generate ultrasound image frame data. If there are multiple acoustic line signals for an observation point Pij at the same position calculated from delay-and-sum processing corresponding to multiple calculation target areas BxI0, an acoustic line signal having a greatest signal intensity, for example, may be stored at the corresponding address in the image memory 106a. In this way, in ultrasound imaging signal sub-frame data corresponding to multiple calculation target areas BxI0, signals with the highest luminance can be used to constitute ultrasound image frame data. However, in THI, it is preferable to use signals with highest luminance after harmonic extraction processing.

Alternatively, a configuration may be used in which a signal obtained by averaging acoustic line signals for an observation point Pij at the same position is stored at a corresponding address. According to this configuration, ultrasound image frame data can be generated that is both influenced by signals with the highest luminance in ultrasound imaging signal sub-frame data and in which noise is suppressed. As above, in THI, it is preferable to perform averaging using signals after harmonic extraction processing.

Synthesized ultrasound image frame data is output to the DSC 107.

### <Operations>

The following describes ultrasound signal processing operations of the ultrasound diagnostic device 100 with the structure described above.

### <Overview of Processing by Ultrasound Diagnostic Device 100>

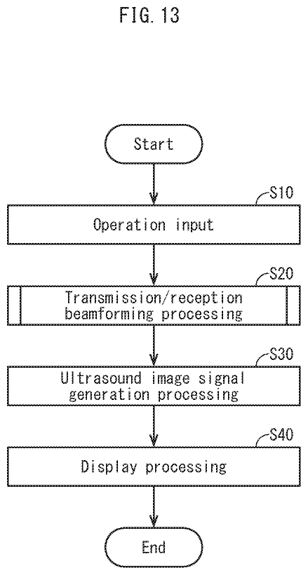

FIG. 13 is a flowchart illustrating an overview of ultrasound signal processing by the ultrasound diagnostic device 100.

First, after ultrasound examination starts, the operation input unit 110 receives various inputs related to the ultrasound diagnostic device 100 such as settings, operations, and the like from a user, and outputs to the controller 109 (step S10).

Next, the transmitter 103 supplies a drive signal pw to transducers in the array Tx of transmission transducers selected from the transducers 101a of the probe 101 (transmission beamforming) to cause the transducers to transmit an ultrasound beam, and the receiver 104 generates acoustic line signals from electric signals obtained by the transducers 101a based on reflected waves received by the probe 101 (reception beamforming), and outputs to the ultrasound imaging signal generator 105 (step S20). The receiver 104 generates acoustic line signal sub-frame data for the calculation target areas BxI0 set, and sequentially outputs to the ultrasound imaging signal generator 105.

Next, the ultrasound imaging signal generator 105 extracts harmonic components from the acoustic line signal sub-frame data corresponding to the calculation target areas BxI0 and output from the receiver 104 to generate broadband acoustic line signals, executes envelope detection, logarithmic compression, and the like to perform luminance conversion, and subject resulting luminance signals to coordinate conversion in an orthogonal coordinate system to generate ultrasound imaging signal sub-frame data. Further, the imaging signal synthesizer 106 synthesizes ultrasound imaging signal sub-frame data corresponding to the calculation target areas BxI0 based on positions of observation points to generate ultrasound image frame data, and outputs to the DSC 107 (step S30).

Finally, the DSC 107 creates a display image including an ultrasound image based on ultrasound image frame data and outputs to the display 108. The display 108 displays a display image and the ultrasound signal processing operation ends (step S40).

### (Transmission and Reception Beamforming)

The following describes details of processing in step S20.

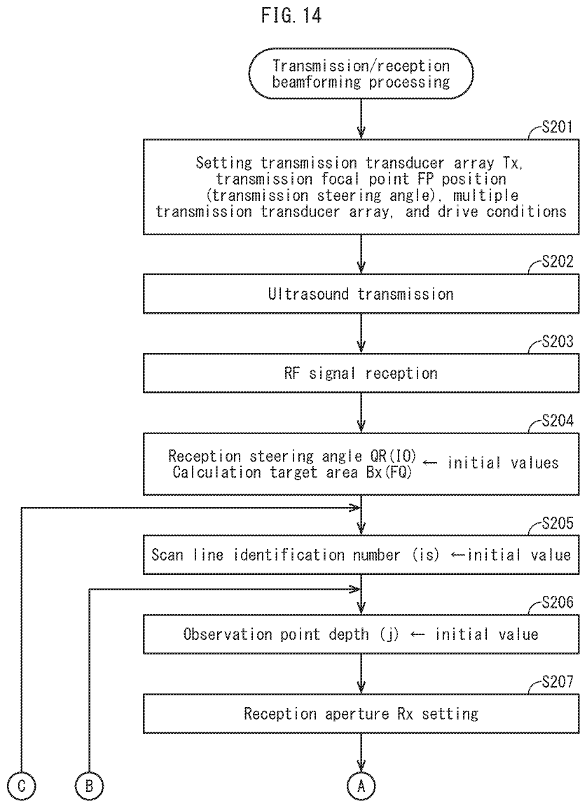

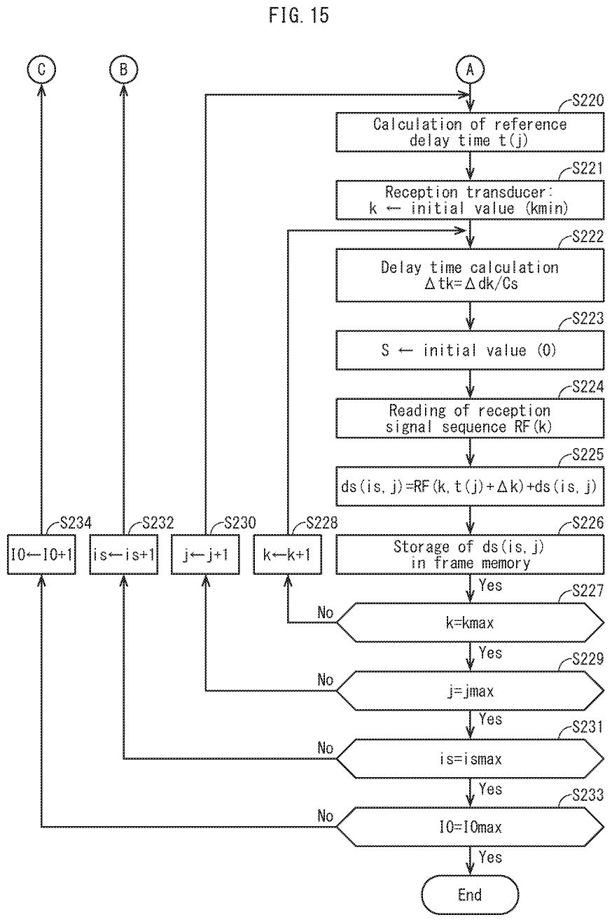

FIG. 14 and FIG. 15 are flowcharts illustrating details of transmission and reception beamforming (step S20 of FIG. 13).

According to the present embodiment, array centers of the array Tx of transmission transducers and the reception aperture Rx coincide in BxC, but BxL and BxR have array centers at positions different from the center of the array Tx, or the array centers may be adjusted according to positions of the observation points Pij. In a calculation target area Bx corresponding to an analysis target range in a subject, when an identification number in the azimuth direction of a scan line intersecting one or more transmission focal points FP is “is” and an index corresponding to a depth direction coordinate Y is “j”, observation points P(is,j) positioned on a scan line (is) are set in order to calculate acoustic line signals.

First, the transmitter 103 acquires a transmission control signal from the controller 109 and sets transmission conditions (step S201). The transmission control signal includes information indicating the array Tx of transmission transducers, position of the transmission focal point FP, drive conditions, and the like.

Next, in step S202, the transmitter 103 performs transmission processing (a transmission event) supplying a drive signal for causing transmission of an ultrasound beam to each transducer included in the array Tx of transmission transducers among the transducers 101a of the probe 101. More specifically, the transmitter 103 supplies different drive signals pw1, pw2, pw3 to the arrays Tx1, the arrays Tx2, and the array Tx3, respectively, causing transmission of the ultrasound beam UsIn from the array Tx3 whose position in the azimuth direction overlaps with the transmission focal point FP, transmission of the ultrasound beams UsO1 from the arrays Tx1 that sandwich the array Tx1 in the azimuth direction, and transmission of the ultrasound beams UsO2 from the arrays Tx2 that sandwich a range including the arrays Tx1 and the array Tx3 in the azimuth direction.

Next, in step S203, the input unit 1041 generates reception signals (RF signals) based on electric signals obtained from reception of ultrasound reflected waves by the probe 101 and outputs to the reception signal storage 1042, which stores the reception signals.

Next, in step S204, the reception aperture setting unit 10431 in the delay-and-sum unit 1043 sets a reception steering angle θR(I0) and a calculation target area Bx(I0) (where I0=1 to Imax; I0 is a natural number; and Imax is 2 or more), and sets the reception steering angle θR(I0) and the calculation target area Bx(I0) to initial values of 1 (step S204). Then, an identification number “is” in the azimuth direction of a scan line intersecting a transmission focal point FP set in the calculation target area Bx(I0) is set to an initial value (step S205), and an index “j” indicating a depth direction coordinate Y of an observation point P(is,j) that is a first calculation target is set to an initial value (step S206). Next, an array of transducers that constitute a reception aperture Rx are set, based on the scan line, or an area center line CL of the calculation target area Bx(I0), or position of the observation point P(is,j) as per Embodiment 2, described later (step S207). The reception aperture Rx may be set symmetrically with respect to a scan line intersecting the observation point P(is,j), for example.

Next, the delay time calculator 10432 calculates a reference arrival time t(j) (step S220). The reference arrival time t(j) is a time required for an ultrasound wave to travel to the observation point P(is,j) from a reception transducer Rw positioned at a center of the reception aperture Rx and back to the reception transducer Rw.

Next, an index k for identifying reception transducers Rw in the reception aperture Rx is set to an initial value (step S221). According to the present embodiment, a minimum value kmin of the reception transducers Rw included in the reception aperture Rx (from kmin to kmax) is set as the initial value.

Next, the delay time calculator 10431 calculates a delay time Δtk for a reflected wave to arrive at the reception transducer Rwk from the observation point P(is,j). More specifically, the delay time calculator 10431 geometrically calculates a length of a path from the observation point P(is,j) to the reception transducer Rwk, based on information indicating position of the reception transducer Rwk and information indicating position of the observation point P(is,j). Then, differences Δdk in path length from the observation point P(is,j) to each reception transducer Rwk are each divided by a sound velocity value Cs, to calculate delay times Δtk for a reflected wave to arrive at each of the reception transducers Rwk from the observation point P(is,j).

Next, the delay processor 10433 sets a delay time application count S to an initial value of 0 (step S223), and reads a reception signal sequence RF(k) from the reception signal storage 1042 (step S224), and specifies a reception signal value RF(k,t(j)+Δtk) in the reception signal sequence RF(k), and calculates a sum of the reception signal value RF(k,t(j)+Δtk) and an acoustic line signal ds(is,j) stored in an addition register (step S225), and stores the new value of the acoustic line signal ds(is,j) in the addition register (step S226). In a first iteration, ds(is,j)=0, and RF(k,t(j)+Δtk) is set in the addition register.

Next, it is determined whether or not the index k identifying the reception transducer Rw is a maximum value kmax (step S227). If k is not kmax, k is incremented by 1 (step S228) and processing returns to step S222. If k is kmax, i.e., a maximum value of reception transducers Rw in the reception aperture Rx, calculation of acoustic line signals dS(is,j) for the observation point P(is,j) has ended, and it is determined whether or not j is a maximum value jmax (step S229). If j is not a maximum value jmax, j is incremented by 1 (step S230) and processing returns to step S220. If j is jmax, calculation of acoustic line signals dS(is,j) for all observation points P(is,j) on a scan line (is) has ended, and it is determined whether or not is is a maximum value ismax (step S231). Next, if is is not a maximum value ismax, is is incremented by 1 (step S232) and processing returns to step S206, If is is ismax, calculation of acoustic line signals dS(is,j) for observation points P(is,j) on all scan lines (is) in a calculation target area Bx has ended, and it is determined whether or not an index I0 of the reception steering angle θR and the calculation target area Bx(I0) is a maximum value I0max (step S233). Next, if I0 is a maximum value I0max, I0 is incremented by 1 (step S234) and processing returns to step S205. If I0 is I0max, calculation of acoustic line signals dS(is,j) for all observation points P(is,j) in all calculation target areas Bx has ended, and processing ends.

### <Generation of Ultrasound Imaging Signals According to Ultrasound Diagnostic Device 100>

The following describes structure and operations of the ultrasound diagnostic device 100 pertaining to Embodiment 1, where the array Tx of transmission transducers is gradually moved one transducer at a time in the azimuth direction, while performing transmission and reception M times, where M corresponds to the number of transducers 101a of the probe 101.

Ultrasound diagnostic devices typically have a structure such that movement of acoustic lines, that is, scan line sub-scanning, starts with a transmission aperture center at one end of a transducer array, and each ultrasound transmission (transmission event), the array Tx of transmission transducers is moved by one transducer in the azimuth direction until the transmission aperture center reaches the other end of the transducer array, so the number M of transmission events corresponds to the number of the transducers 101a of the probe 101.

According to the ultrasound diagnostic device 100 pertaining to Embodiment 1, the array Tx of transmission transducers is moved gradually by one transducer in the azimuth direction, and the transmission aperture center moves from one end to another end, and the number M of transmission events corresponds to the number of the transducers 101a of the probe 101. However, in the ultrasound diagnostic device 100, while the transmitter 103 has a point in common with typical ultrasound diagnostic devices in that ultrasound transmissions (transmission events) are repeated while gradually moving the array Tx of transmission transducers in the array direction, the number of transmission events to form a frame and the start and end positions of transmission events are different.

FIG. 16 is a schematic diagram illustrating propagation paths of ultrasound beams pertaining to transmission according to the transmitter 103 of the ultrasound diagnostic device 100 pertaining to Embodiment 1. In the example illustrated in FIG. 16, the number M of transducers selected as the array Tx of transmission transducers is the same as the number N of the transducers 101a of the probe 101. According to the ultrasound diagnostic device 100, as illustrated in FIG. 16, ultrasound beams are sequentially transmitted focused on each transmission focal point FP from each array Tx of transmission transducers due to the transmitter 103 selecting, multiple times, the array Tx of transmission transducers from the transducers 101a while gradually moving the array Tx in the azimuth direction, and setting, multiple times, the transmission focal point FP in the azimuth direction corresponding to this selection. At this time, for each ultrasound transmission (transmission event), the array Tx of transmission transducers may be gradually moved one transducer at a time in the azimuth direction, where the number of transmission events may correspond to M+N, where M is the number of the transducers 101a of the probe 101 and N is the number of transmission aperture transducers. In this case, in a first transmission event, only a first transducer among the transducers 101a of the probe 101 (leftmost in the azimuth direction in FIG. 16) is driven, and in an M+Nth transmission event, only an Mth transducer (rightmost in the azimuth direction in FIG. 16) is driven. In the first transmission event, a transducer at one end of the transducers 101a of the probe 101 emits an ultrasound beam towards a transmission focal point FP positioned outside the one end of the probe 101 in the azimuth direction, and in the last transmission event, a transducer at the other end of the probe 101 in the azimuth direction emits an ultrasound beam towards a transmission focal point FP positioned outside the other end of the probe 101 in the azimuth direction.

Accordingly, the BxL and BxR areas can be obtained up to both ends of the transducers in the same way as the BxC area as illustrated in FIG. 12, and overlapping areas are expanded in the azimuth direction. The above sub-scanning method need not always be used, and in a case where time resolution is prioritized over enlargement of the overlapping areas, a user may select to use a typical sub-scanning method, or the above sub-scanning method and a typical sub-scanning method may be automatically switched according to changes in transmission focal depth or display depth.

The receiver 104 generates acoustic line signal sub-frame data dsI0 for the calculation target areas BxI0 each having different reception steering angles θR, based on reception signals obtained based on corresponding ultrasound transmissions. Further, the imaging signal synthesizer 106 synthesizes ultrasound imaging signal frame data based on acoustic line signal sub-frame data dsI0 corresponding to the arrays Tx of transmission transducers and transmission focal points FP to generate ultrasound imaging signal frame data based on each ultrasound transmission, and further synthesizes ultrasound imaging signal frame data based on observation point positions to generate ultrasound imaging signal integrated frame data based on ultrasound transmissions.

The following describes ultrasound signal processing of the ultrasound diagnostic device 100 pertaining to Embodiment 1 with the structure described above.

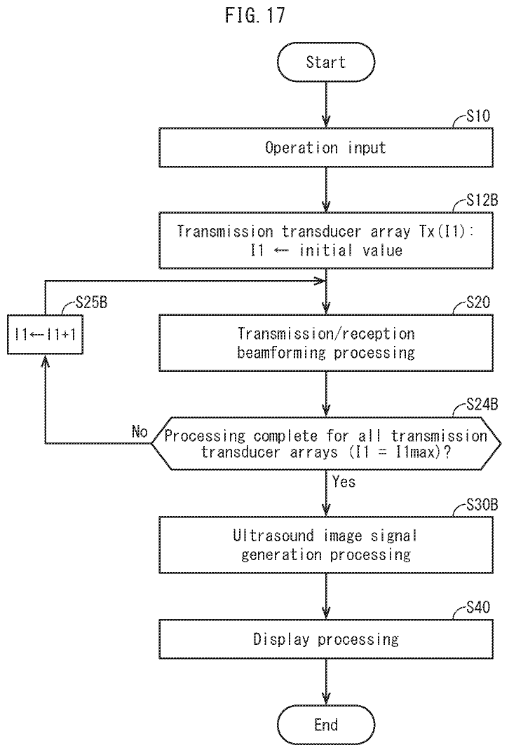

FIG. 17 is a flowchart illustrating processing of the ultrasound diagnostic device 100 when the array Tx of transmission transducers is gradually moved one transducer at a time in the azimuth direction, performing transmission and reception M times, where M corresponds to the number of the transducers 101a of the probe 101. Steps that are different from those in FIG. 13 are denoted by different numbers, and detailed description of identical steps is not repeated here.

First, output of various operation inputs to the controller 109 in step S10 is the same as previously described.

Next, the transmitter 103 sets a position I1 in the azimuth direction of the array Tx of transmission transducers to an initial value (step S12B). At this time, the position of the transmission focal point FP in the azimuth direction is also set corresponding to the position I1 in the azimuth direction of the array Tx of transmission transducers.

Next, transmission and reception beamforming is performed based on the flowcharts illustrated in FIG. 14 and FIG. 15 (step S20). That is, the transmitter 103 causes the array Tx of transmission transducers to transmit an ultrasound beam in a state where the position I1 of the array Tx and the transmission focal point FP are set to initial values, the receiver 104 generates acoustic line signal sub-frame data corresponding to the calculation target areas BxI1 based on obtained reflected waves, and sequentially outputs to the ultrasound imaging signal generator 105.

Next, it is determined whether or not the position I1 in the azimuth direction of the array Tx is a maximum value I1max (step S24B). Then, if I1 is not the maximum value I1max, I1 is incremented by 1 (step S25B), and processing returns to step S20. If I1 is the maximum value I1max, acoustic line signal calculation has ended for all positions I1 in the azimuth direction of the array Tx, and processing proceeds to step S30B.

Next, in step S30B, the ultrasound imaging signal generator 105 generates ultrasound imaging signal sub-frame data from acoustic line signal sub-frame data corresponding to calculation target areas BxI1 and obtained from transmission events each having a different position I1 in the azimuth direction of the array Tx, output from the receiver 104. Further, the imaging signal synthesizer 106 synthesizes the ultrasound imaging signal sub-frame data corresponding to the calculation target areas BxI1, with respect to the positions I1 in the azimuth direction of the array Tx, to generate ultrasound image frame data. Further, the imaging signal synthesizer 106 synthesizes the ultrasound image frame data corresponding to the positions I1 in the azimuth direction of the array Tx to generate ultrasound image integrated frame data, and outputs to the DSC 107.

Finally, in step S40, the DSC 107 creates a display image including an ultrasound image based on the ultrasound image integrated frame data, and causes the display 108 to display same.

According to this processing, the ultrasound diagnostic device 100 generates acoustic line signals for transmission events while gradually moving the array Tx of transmission transducers in the azimuth direction, synthesizes ultrasound image frame data obtained from transmission events, and generates ultrasound image integrated frame data. Thus, for the same observation point, an observation imaging signal can be generated based on reception signals from different positions of the array Tx of transmission transducers, and spatial resolution and signal to noise ratio can be improved. Further, as described above, in the first transmission event, a transducer at one end of the transducers 101a of the probe 101 emits an ultrasound beam towards a transmission focal point FP positioned outside the one end of the probe 101 in the azimuth direction, and in the last transmission event, a transducer at the other end of the probe 101 in the azimuth direction emits an ultrasound beam towards a transmission focal point FP positioned outside the other end of the probe 101 in the azimuth direction. Thus, when an anisotropic specular reflection member is located in a shallow region farther outwards than either end of the transducers 101a of the probe 101, a reflected wave from the anisotropic specular reflection member is effectively received, making it possible to improve visibility of the anisotropic specular reflection member such as a puncture needle.

### <Review>

As described above, the ultrasound diagnostic device 100 pertaining to Embodiment 1 comprises the transmitter 103, the input unit 1041, the delay-and-sum unit 1043, and the imaging signal synthesizer 106. The transmitter 103 selects as an array of transmission transducers, a tertiary transducer array Tx3, two partial primary transducer arrays Tx1 that sandwich the tertiary transducer Tx3 array in the azimuth direction, and two secondary transducer arrays Tx2 that sandwich the partial primary transducer arrays Tx1 in the azimuth direction, and causes transmission of an ultrasound beam from the array of transmission transducers such that a portion of the ultrasound beam from the tertiary and secondary transducer arrays Tx3, Tx2 has a larger signal intensity in a high frequency band than a portion of the ultrasound beam from the partial primary transducer arrays Tx1. The delay-and-sum unit 1043 sets calculation target areas that include different areas in the azimuth direction, and with respect to observation points in the calculation target areas, executes delay-and-sum processing to generate acoustic line signal sub-frame data and the like.

According to this structure, while an ultrasound beam UsO2 having a large signal intensity in a high frequency band is emitted from the arrays Tx2, reflected waves from observation points Pij are received from a wide viewing angle by a reception aperture Rxq corresponding to a reception steering angle θRC of a calculation target area Bxq. Therefore, for example, reflected waves from an anisotropic reflection site located in a shallow region or a peripheral region thereof, such as a high-angle puncture needle shaft, a longitudinal tissue boundary, an anterior talofibular ligament, or the like, can be received with higher probability at any of the reception apertures Rx of the calculation target areas Bxq, and reflected waves can be received by the reception aperture Rxq with the highest sensitivity.

As a result, in an inexpensive device that does not require complex transmission control, visibility of an anisotropic high-reflection member in a shallow peripheral region of ultrasound irradiation is improved, and rendering of a high-angle anisotropic reflection site can be improved over conventional technology. In other words, anisotropic sites and puncture needles, which were difficult to render conventionally and depended on user skill to render, can be observed clearly by even an unskilled person without requiring complicated transmission control, with a relatively inexpensive device, and without increasing the number of transmissions or decreasing video performance, so that diagnostic accuracy can be improved, and safety and workability of guided procedures can be improved.

### Modification 1

Although the ultrasound diagnostic device 100 pertaining to Embodiment 1 has been described, the present disclosure is not limited to the embodiment described above aside from essential characteristic components thereof. The following describes a modification of the ultrasound diagnostic device 100 as an embodiment.

According to the ultrasound diagnostic device 100 pertaining to Embodiment 1, as illustrated in FIG. 5, an ultrasound beam is transmitted focused on a specific site in a subject corresponding to the transmission focal point FP from the array Tx of transmission transducers, and based on obtained reflected wave signals, acoustic line signal sub-frame data dsI0 is generated for calculation target sites BxI0 for which reception steering angles θR are different.

In contrast, according to the ultrasound diagnostic device pertaining to Modification 1, multiple transmission focal points FP with different positions in the azimuth direction are set, and multiple transmission events cause ultrasound beams to be transmitted from the same array Tx of transmission transducers to specific sites in a subject corresponding to the transmission focal points FP, changing the transmission focal point FP each time.

FIG. 18A, 18B, 18C are schematic diagrams illustrating ultrasound beam propagation paths pertaining to transmission according to the transmitter 103 of an ultrasound diagnostic device pertaining to Modification 1. As illustrated in FIG. 18A, 18B, 18C, according to the ultrasound diagnostic device pertaining to Modification 1, ultrasound beams are transmitted with different steering angles of θTL, θTC, θTR, from the same array Tx of transmission transducers while changing positions in the azimuth direction of the transmission focal point FP. The transmission steering angles θTL, θTR may be, for example, +/−10°, and the transmission steering angle θTC may be 0°. Other structure and operations related to transmission are the same as those of the ultrasound diagnostic device 100 pertaining to Embodiment 1.

According to this configuration, the number of directions covered by ultrasound irradiation areas AxL, AxC, AxR obtained from transmission events corresponding to transmission steering angles θTL, θTC, θTR increases.

Further, according to the ultrasound diagnostic device pertaining to Modification 1, based on received signals obtained from the transmission events with different transmission steering angles θT, further acoustic line signal sub-frame data dsI0 is generated for calculation target areas BxI0 having different reception steering angles θR. In each transmission event, the reception steering angle θR set in sub-frame reception is set to be adjusted according to each transmission steering angle θT. Thus, the changes in range and number of direction of the reception steering angle θR between transmission events are increased when compared to Embodiment 1.

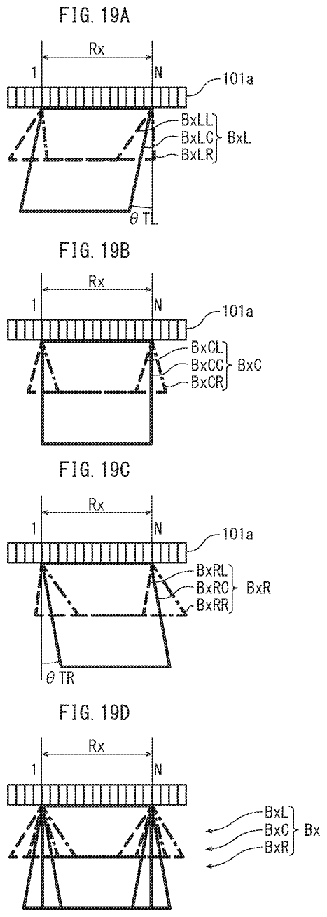

FIG. 19A, 19B, 19C, 19D are schematic diagrams for explaining generation of ultrasound image frame data by the imaging signal synthesizer 106 of an ultrasound diagnostic device pertaining to Modification 1.

As illustrated in FIG. 19A, based on reception signals obtained from a transmission event in which the transmission steering angle θT is θTL, as per the ultrasound diagnostic device 100 of Embodiment 1, the imaging signal synthesizer 106 sets calculation target areas BxLL, BxLC, BxLR, and synthesizes ultrasound imaging signal sub-frame data using positions of observation points as a reference based on acoustic line signal sub-frame data obtained from delay-and-sum processing with respect to each calculation target area, in order to generate ultrasound imaging signal frame data corresponding to the calculation target area BxL.

Similarly, as illustrated in FIG. 19B, based on reception signals obtained when the transmission steering angle θT is 0, the imaging signal synthesizer 106 sets calculation target areas BxCL, BxCC, BxCR, and synthesizes ultrasound imaging signal sub-frame data based on acoustic line signal sub-frame data obtained from delay-and-sum processing with respect to each calculation target area, in order to generate ultrasound imaging signal frame data corresponding to the calculation target area BxC.

Further, as illustrated in FIG. 19C, based on reception signals obtained when the transmission steering angle θT is θTR, the imaging signal synthesizer 106 sets calculation target areas BxRL, BxRC, BxRR, and synthesizes ultrasound imaging signal sub-frame data based on acoustic line signal sub-frame data obtained from delay-and-sum processing with respect to each calculation target area, in order to generate ultrasound imaging signal frame data corresponding to the calculation target area BxR.

Then, as illustrated in FIG. 19D, the imaging signal synthesizer 106 synthesizes ultrasound imaging signal frame data corresponding to the calculation target areas BxL, BxC, BxR using positions of observation points as a reference to generate ultrasound imaging signal integrated frame data corresponding to all calculation target areas Bx.

The following describes ultrasound signal processing of the ultrasound diagnostic device pertaining to Modification 1 as described above.

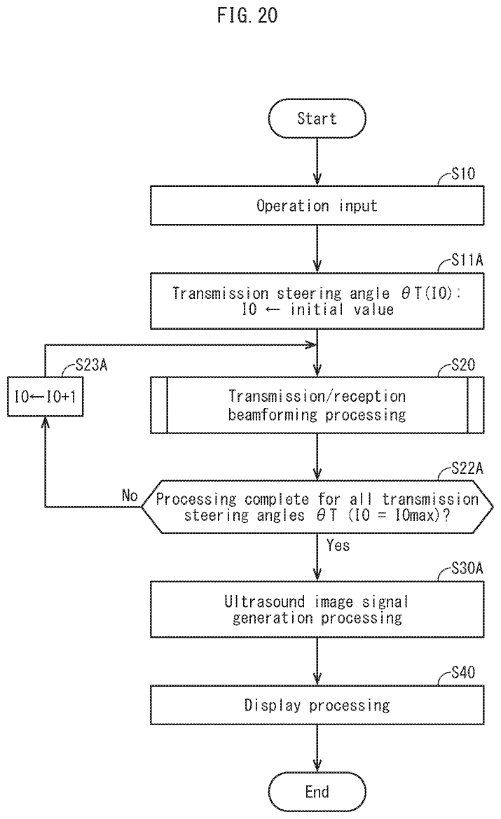

FIG. 20 is a flowchart illustrating processing by the ultrasound diagnostic device pertaining to Modification 1. Steps that are different from those in FIG. 13 are denoted by different numbers, and detailed description of identical steps is not repeated here.

First, output of various operation inputs to the controller 109 in step S10 is the same as previously described.

Next, the transmitter 103 sets the transmission steering angle OT(I0) to an initial value (step S11A).

Next, transmission and reception beamforming is performed based on the flowchart illustrated in FIG. 14 and FIG. 15 (step S20). That is, the transmitter 103 causes transmission of an ultrasound beam from transducers in the array Tx in a state where the transmission steering angle OT(I0) is set to the initial value, and the receiver 104 generates acoustic line signal sub-frame data corresponding to the calculation target areas BxI0 based on obtained reflected waves, and outputs sequentially to the ultrasound imaging signal generator 105.

Next, it is determined whether or not the transmission steering angle OT(I0) is a maximum value I0max (step S22A). Then, if I0 is not the maximum value I0max, I0 is incremented by 1 (step S23A), and processing returns to step S20. If I0 is the maximum value I0max, acoustic line signal calculation has ended for all transmission steering angles θT, and processing proceeds to step S30A.

Next, in step S30A, the ultrasound imaging signal generator 105 generates ultrasound imaging signal sub-frame data from acoustic line signal sub-frame data corresponding to calculation target areas BxI0 and obtained with transmission steering angles θT(I0), output from the receiver 104. Further, the imaging signal synthesizer 106 synthesizes the ultrasound imaging signal sub-frame data corresponding to the calculation target areas BxI0, with respect to the transmission steering angles θT(I0), to generate ultrasound image frame data. Further, the imaging signal synthesizer 106 synthesizes the ultrasound imaging signal frame data corresponding to the transmission steering angles θT(I0) to generate ultrasound imaging signal integrated frame data, and outputs to the DSC 107.

Finally, in step S40, the DSC 107 creates a display image including an ultrasound image based on the ultrasound imaging signal integrated frame data, and causes the display 108 to display same.

According to the ultrasound diagnostic device pertaining to Modification 1, an ultrasound beam having a high harmonic generation capability in a shallow region (UsIn, UsO2) can be transmitted with an even larger transmission steering angle θT, and due to the increase in transmission steering angle θT, delay-and-sum processing can be performed by using an even larger reception steering angle θR. Thus, when compared to Embodiment 1, an absolute value of the viewing angle of reception in delay-and-sum processing is increased, and it is possible to increase the probability of a reflected wave from an anisotropic reflection site such as a tendon, a specular reflection member such as a puncture needle, or the like in a shallow region or peripheral region thereof being captured by a reception aperture Rxq, increasing visibility, as well as increasing the number of sub-frames constituting integrated frame data, increasing image homogeneity.

### Embodiment 2

According to the ultrasound diagnostic device 100 pertaining to Embodiment 1, the reception aperture setting unit 10431 of the delay-and-sum unit 1043 sets a reception aperture Rx with respect to an observation point Pij such that a reception acoustic line intersects the observation point Pij and has a starting point at the array Tx3 or one of the arrays Tx2—that is, in UsIn, UsO2, a synthesized wavefront propagation angle and a reception acoustic line angle are the same. However, a method of selecting a reception aperture Rx is not limited to this, and a reception aperture Rx may be set in a different way.

As an example of reception aperture Rx setting, acoustic line signals are generated by moving reception focal positions according to transmission wavefront progression in the UsIn, UsO2 areas, as per Embodiment 1, but a center of the reception aperture Rx is not fixed as an approximate center of the array Tx1 or one of the arrays Tx3, but instead all acoustic line signals are received in a same direction with respect to observation points Pij set in the UsIn, UsO2 areas. In other words, with respect to transmission with a steering angle of 0°, reception is performed with a steering angle of 0° for all observation points set in the areas UsIn, UsO2, and with respect to transmission with a steering angle of x°, reception is performed with a steering angle of x°. That is, a center of a reception aperture Rx of the area UsIn does not move, but a center of a reception aperture Rx with respect to observation points set in the area UsO2 moves in the azimuth direction according to transmission wavefront progression. Directions of transmission and reception for UsIn are the same, but directions of transmission and reception for UsO2 are not the same. Compared to Embodiment 1, angles corresponding to anisotropic high-reflection members and sites are reduced by half, but received signals have short propagation paths and are less affected by attenuation, and artifacts due to grating lobes are less likely to occur, and therefore it is preferable to select appropriate values according to probe properties and purpose to be emphasized.

The ultrasound diagnostic device pertaining to Embodiment 2 is different from the ultrasound diagnostic device 100 pertaining to Embodiment 1 in a method of selecting reception aperture Rx by the reception aperture setting unit 10431. Other structure is same as that of the ultrasound diagnostic device 100 illustrated in FIG. 2, 3, 8, and therefore detailed description thereof is not repeated here.

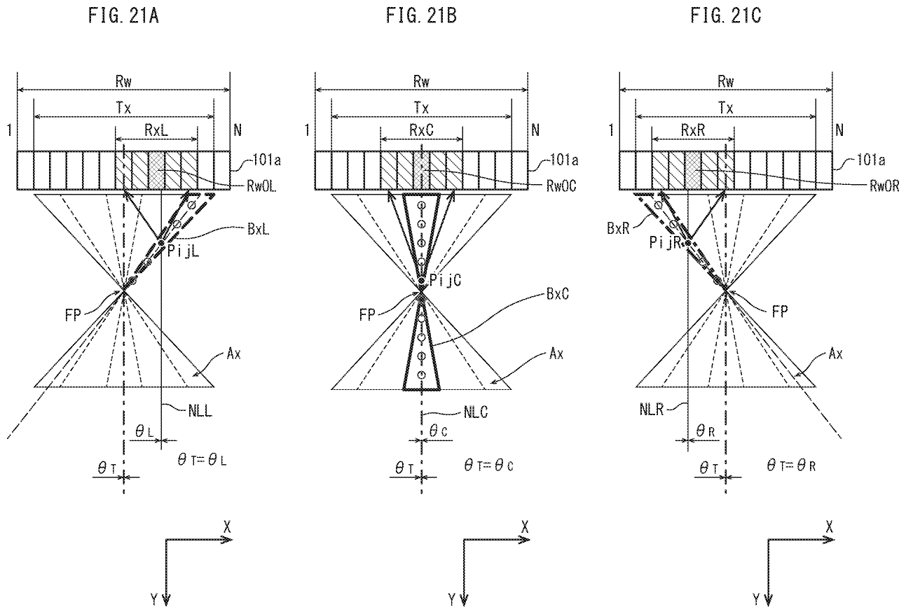

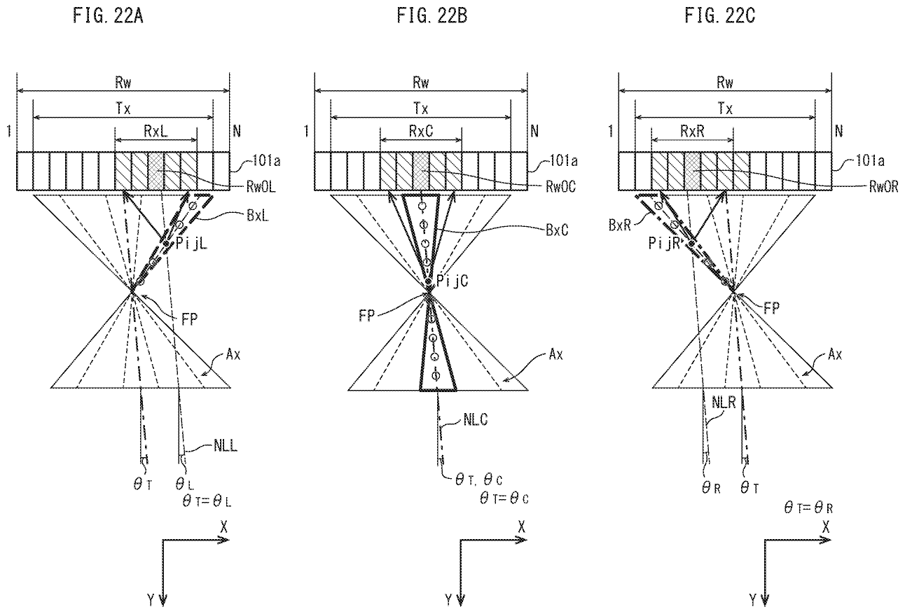

FIG. 21A, 21B, 21C are schematic diagrams for explaining acoustic line signal generation for observation points Pij by the delay-and-sum unit 1043 of the ultrasound diagnostic device pertaining to Embodiment 2. FIG. 22A, 22B, 22C are schematic diagrams for explaining acoustic line signal generation for observation points Pij by the delay-and-sum unit 1043 when a transmission steering angle θT is added, with respect to the ultrasound diagnostic device pertaining to Embodiment 2. In FIG. 21A, 21B, 21C, 22A, 22B, 22C, the calculation target areas BxL, BxC, BxR and the reception steering angles are the same as the calculation target areas BxL, BxC, BxR and the reception steering angles θRL, θRC, θRR illustrated in FIG. 9A, 9B, 8C, 10A, 10B, 10C, respectively.

As illustrated in FIG. 21A, 21B, 21C, 22A, 22B, 22C, when the transmission steering angle θT is used as a reference angle, transducer arrays of the reception apertures RxL, RxC, RxR in delay-and-sum processing for observation points PijL, PijC, PijR in the calculation target areas BxL, BxC, BxR are selected as follows. Angles θL, θC, θR from a vertical direction of straight lines NLL, NLC, NLR drawn from array centers of reception apertures RxL, RxC, RxR to observation points PijL, PijC, PijR (reception direction angles) are selected to be the same as θT, which is a reference angle. When the transmission steering angle θT is 0°, as in the example illustrated in FIG. 21A, 21B, 21C, straight lines NLL, NLC, NLR that link observation points PijL, PijC, PijR to nearest transducers RwOL, RwOL, RwOR, respectively, are selected as centers of reception apertures RxL, RxC, RxR. Further, for the reception apertures RxL, RxC, RxR, a weighting sequence may be adopted in which reception transducers with a shorter distance to the observation point Pij are assigned a greater weight (reception apodization).

According to this configuration of the ultrasound diagnostic device pertaining to Embodiment 2, the reception aperture setting unit 10431 selects a reception aperture Rx transducer array such that an array center coincides with a transducer that is spatially closest to an observation point Pij, then performs reception beamforming using the reception aperture Rx that is symmetrical about the observation point Pij, based on the observation point Pij, regardless of the transmission event. Thus, even when considering that attenuation increases with ultrasound propagation path distance to an observation point Pij, reflected waves from the observation point Pij can be received with the least possible propagation attenuation effect. Further, by receiving reflected waves from an observation point Pij in the UsO2 area, where steering is applied in a transmission propagation direction and a grating lobe is likely to occur, in a direction different from the steering angle at a reception aperture center, overlap between transmission and reception grating lobes can be reduced, reducing artifacts and improving signal quality. In addition, as per Embodiment 1, by substantially increasing the number of transmission directions and angles, reflected waves from anisotropic reflection members in a shallow region can be effectively received, improving visibility of specular reflection members such as puncture needles.

### Modification 2

The following describes a modification of the ultrasound diagnostic device pertaining to Embodiment 2. According to the ultrasound diagnostic device pertaining to Embodiment 2, from an array of transmission transducers, the transmitter 103 selects a tertiary array Tx3 of transducers whose position in the azimuth direction overlaps with the position in the azimuth direction of the transmission focal point, two partial primary arrays Tx1 of transducers that sandwich the tertiary array Tx3 in the azimuth direction, and two secondary arrays Tx2 of transducers that sandwich the transducers of the partial primary arrays Tx1 and the tertiary array Tx3 in the azimuth direction. Further, the transmitter 103 causes transmission of an ultrasound beam having a higher signal intensity in a high frequency band from the tertiary array Tx3 and the secondary arrays Tx2 than from the partial primary arrays Tx1. According to the ultrasound diagnostic device pertaining to Modification 2, in addition to the configuration according to Embodiment 2, the transmitter 103 selects two quaternary arrays Tx4 of transducers positioned between the partial primary arrays Tx1 and the secondary arrays Tx2 in the azimuth direction, and causes the quaternary arrays Tx4 of transducers to not transmit an ultrasound beam.

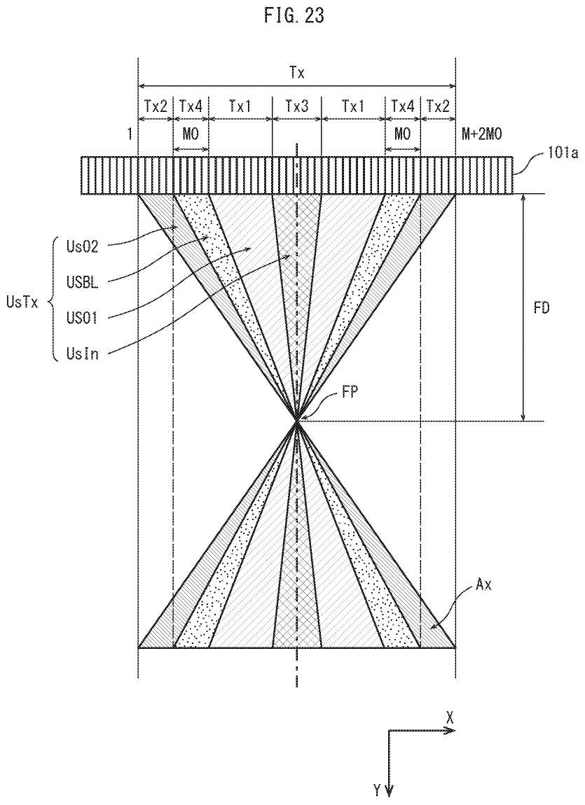

FIG. 23 is a schematic diagram illustrating ultrasound beam propagation paths pertaining to transmission according to the transmitter 103 of an ultrasound diagnostic device pertaining to Modification 2. As illustrated in FIG. 23, according to Modification 2, the transmitter 103 selects two quaternary arrays of transducers (also referred to as the “arrays Tx4”) between the arrays Tx1 and the arrays Tx2. Then, the ultrasound beams UsIn, UsO2 are transmitted from the array Tx3 and the arrays Tx2, and the ultrasound beams UsO1 from the arrays Tx1, and while drive signals are generated and supplied to the array Tx3 and the arrays Tx2 that have different frequency distribution to the drive signals generated and supplied to the arrays Tx1, the arrays Tx4 are not driven to transmit an ultrasound beam.

According to this configuration, the number of transducers included in the array Tx increases by a number corresponding to twice the number of transducers in one of the arrays Tx4. However, since an ultrasound beam is not transmitted from the arrays Tx4, energy consumption due to application of the drive signal pw does not increase. Thus, as illustrated in FIG. 23, when a width in the azimuth direction of the array Tx4 is M0, increases in energy consumption due to application of the drive signal pw and transducer heat generation are suppressed and a difference in angles between the UsIn area and the UsO2 areas can be increased. As a result, a substantial viewing angle that can be received by a reception aperture in delay-and-sum processing is expanded, and reflected waves from an anisotropic high-reflection member in a shallow region or peripheral region thereof are more effectively received, improving visibility of a specular reflection member such as a puncture needle.

### Other Modifications Pertaining to Embodiments 1 and 2

Although ultrasound diagnostic devices pertaining to Embodiments 1 and 2 have been described, the present disclosure is not limited to the embodiments described above aside from essential characteristic components thereof. For example, the scope of the present disclosure also includes embodiments derived from modifications made by a person skilled in the art to Embodiments 1 and 2 and Modifications thereof, and embodiments derived from any combination of components and functions described that does not depart from the spirit of the present invention.

The following describes further modifications of Embodiments 1 and 2.

### Modification 3

According to the ultrasound diagnostic devices pertaining to Embodiments 1 and 2, from an array of transmission transducers, the transmitter 103 selects a tertiary array Tx3 of transducers whose position in the azimuth direction overlaps with the position in the azimuth direction of the transmission focal point, two partial primary arrays Tx1 of transducers that sandwich the tertiary array Tx3 in the azimuth direction, and two secondary arrays Tx2 of transducers that sandwich the transducers of the tertiary array Tx3 and the partial primary arrays Tx1 in the azimuth direction. Further, the transmitter 103 causes transmission of an ultrasound beam having a higher signal intensity in a high frequency band from the tertiary array Tx3 and the secondary arrays Tx2 than from the partial primary arrays Tx1. However, frequency properties of ultrasound beams transmitted from the secondary arrays Tx2 are not limited to this example.

More specifically, the ultrasound diagnostic device according to Modification 3 includes the example of partial transducer array transmission such that the ultrasound beam UsO3 with a high signal intensity in a high frequency band from the secondary arrays Tx2 irradiates a peripheral area in the ultrasound irradiation area Ax in the shallow region where attenuation in the high frequency band is small.

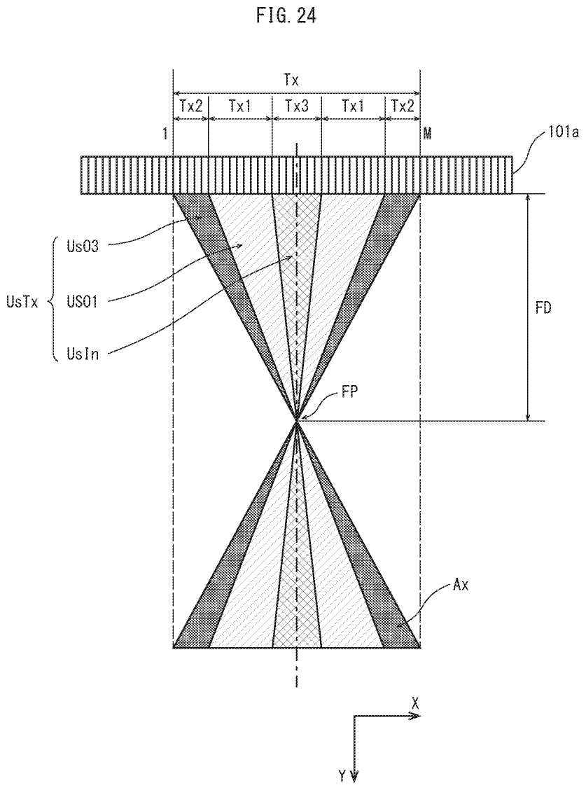

FIG. 24 is a schematic diagram illustrating ultrasound beam propagation paths pertaining to transmission according to the transmitter 103 of an ultrasound diagnostic device pertaining to Modification 3. As illustrated in FIG. 24, according to the ultrasound diagnostic device pertaining to Modification 3, from an array of transmission transducers, the transmitter 103 selects the tertiary array Tx3, the partial primary arrays Tx1 that sandwich the tertiary array Tx3 in the azimuth direction, and the secondary arrays Tx2 that sandwich the transducers included in the tertiary array Tx3 and the partial primary arrays Tx1. Further, the transmitter 103 causes transmission of the ultrasound beam UsIn from the tertiary array Tx3, an ultrasound beam UsO1 from the partial primary arrays Tx1, and from the secondary arrays Tx2, an ultrasound beam UsO3 with a greater signal strength than ultrasound from the tertiary array Tx3 in a frequency band higher than that of a center frequency and a smaller signal strength than ultrasound from the tertiary array Tx3 and the partial primary arrays Tx1 in a frequency band lower than the center frequency.

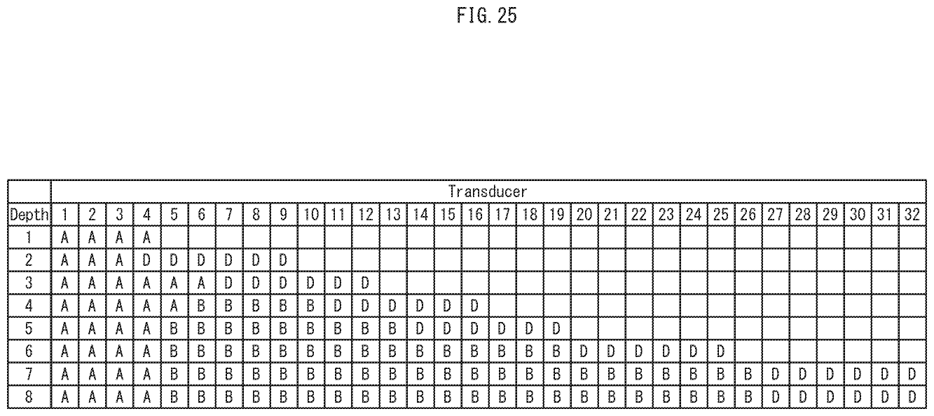

FIG. 25 is a schematic diagram illustrating a relationship between depth FD of the transmission focal point FP and drive signal of transmission transducers with respect to ultrasound beams pertaining to transmission caused by the transmitter 103 of the ultrasound diagnostic device pertaining to Modification 3, in which rows and columns have the same meanings as in FIG. 6. According to the ultrasound diagnostic device pertaining to Modification 3, as illustrated in FIG. 25, when the depth of the transmission focal point FP is “3” or greater, the transmitter 103 selects the array Txq of transmission transducers from the transducers 101a (where q=1 to qmax, q is a natural number, and qmax is 3 or more). Then, drive signals pwq (where q=1 to qmax) in a frequency distribution corresponding to “A, B, D” are individually supplied to transmission transducers of the array Txq, causing transmission of an ultrasound beam focused on the transmission focal point FP from the array Txq of the transmission transducers.

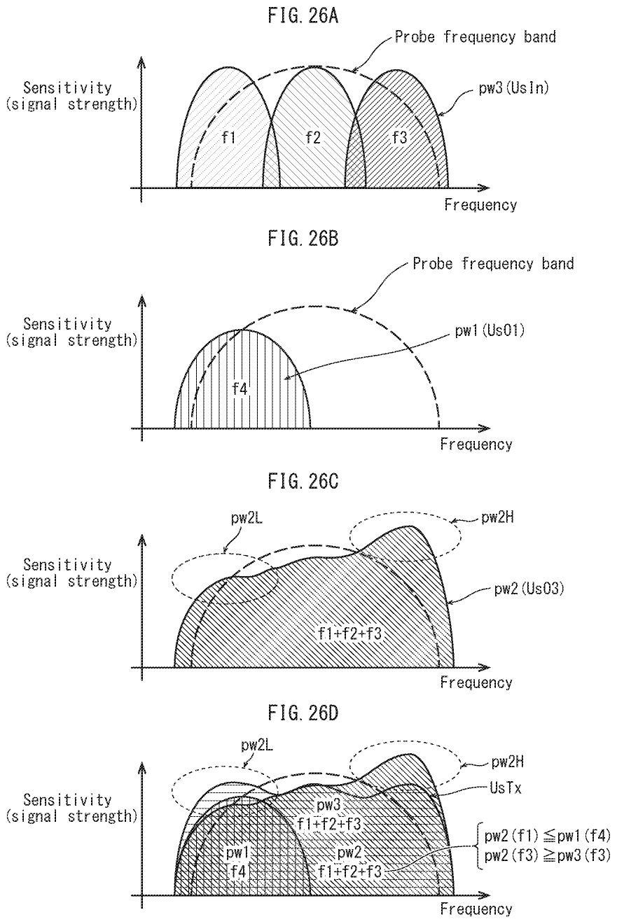

FIG. 26A, 26B, 26C, 26D are schematic diagrams illustrating ultrasound beam frequency distributions pertaining to transmission according to the transmitter 103, where FIG. 26A illustrates UsIn frequency distribution and FIG. 26B illustrates UsO1 frequency distribution, and these distributions correspond to Embodiment 1 as illustrated in FIG. 7A and FIG. 7B, respectively. Further, FIG. 26C illustrates UsO3 frequency distribution, and FIG. 26D illustrates a combination of UsIn, UsO1, UsO3 frequency distribution.

The frequency distributions in FIG. 26A, 26B are the same as those of Embodiment 1 illustrated in FIG. 7A, 7B, and therefore description is repeated here.

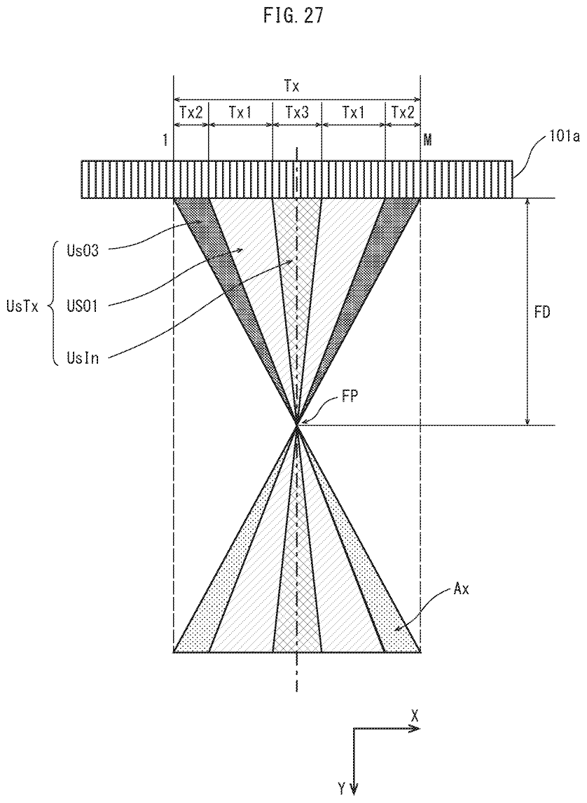

FIG. 27 is a schematic diagram for explaining attenuation of an ultrasound beam UsO3 transmitted from the secondary arrays Tx2 according to the transmitter 103 of an ultrasound diagnostic device pertaining to Modification 3.

As illustrated in FIG. 26C, 26D, frequency distribution of a drive signal pw2 supplied to the arrays Tx2 has a higher signal strength (pw2H) in a fundamental wave f3 frequency component than frequency distribution of a drive signal pw3 supplied to the array Tx3, which includes fundamental wave f1, f2, f3 frequency components. Therefore, according to the ultrasound diagnostic device pertaining to Modification 3, by applying the drive signal pw2 to the arrays Tx2 and the drive signal pw3 to the array Tx3, an ultrasound beam UsO3 with a larger signal strength in a high-frequency band than the ultrasound beam UsO2 of Embodiment 1 can be transmitted from the arrays Tx2.

By transmitting the ultrasound beam UsO3 with a large signal intensity in a high-frequency band from the arrays Tx2, a high-frequency component can be sufficiently generated for a strong signal even in the UsO3 area in which the ultrasound beam steering angle is large and directivity of the transducers 101a causes a tendency towards a decrease in beam intensity when compared to forwards transmission perpendicular to the azimuth direction, increasing resolution in rendering a shallow region. Further, effective reception of reflected waves from anisotropic reflection members that often exist in shallow regions is improved, improving visibility of specular reflection members such as puncture needles.

On the other hand, as illustrated in FIG. 26C, 26D, frequency distribution of the drive signal pw2 supplied to the arrays Tx2 has a lower signal strength (pw2L) in a fundamental wave f1 frequency component than frequency distribution of the drive signal pw3 supplied to the array Tx3, which includes fundamental wave f1, f2, f3 frequency components. Therefore, according to the ultrasound diagnostic device pertaining to Modification 3, an ultrasound beam UsO3 with a smaller signal strength in a low-frequency band than the ultrasound beam UsO2 of Embodiment 1 can be transmitted from the arrays Tx2.

Similarly, as illustrated in FIG. 26C, 26D, frequency distribution of the drive signal pw2 supplied to the arrays Tx2 has a lower signal strength (pw2L) in a fundamental wave f4 frequency component than frequency distribution of the drive signal pw1 supplied to the arrays Tx1, which includes the fundamental wave f4 frequency component. Therefore, according to the ultrasound diagnostic device pertaining to Modification 3, by applying the drive signal pw2 to the arrays Tx2, the ultrasound beam UsO3 with a smaller signal strength in a low-frequency band than the ultrasound beam UsO1 of Embodiment 1 can be transmitted from the arrays Tx2.

An ultrasound beam including a fundamental wave in a low frequency band has high depth penetration, and forms a beam profile in a deeper region based on a low frequency fundamental wave component. Although a beam profile in a deeper region is preferably a single peak shape, according to the ultrasound beam UsO2 transmitted from the arrays Tx2 of Embodiment 1, intensity of an ultrasound beam in a low frequency range tends to be higher in a peripheral area than a central area in an ultrasound irradiation area Ax in the deeper region, and in some cases the ultrasound beam may split in the deeper region.

According to the ultrasound diagnostic device pertaining to Modification 3, the ultrasound beam UsO3 transmitted from the arrays Tx2 has a higher depth penetration smaller signal strength in the low-frequency band than the ultrasound beam UsO2 transmitted from the arrays Tx2 of Embodiment 1, and therefore the ultrasound beam UsO3 from the arrays Tx2 do not reach a deeper region due to attenuation, and therefore it is possible to suppress the occurrence of a split in the ultrasound beam in the deeper region, as illustrated in FIG. 27.

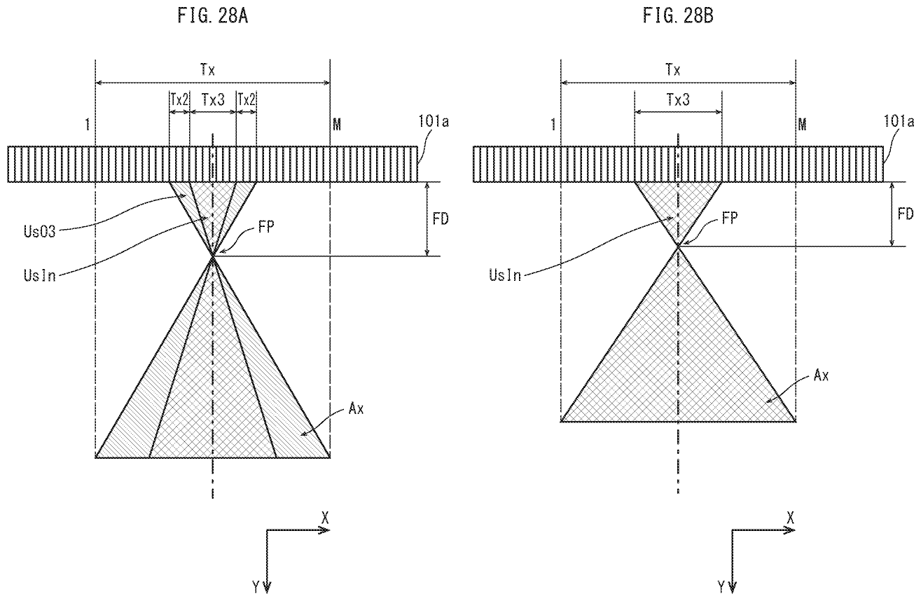

The following is a description of transmission processing by the transmitter 103 of the ultrasound diagnostic device pertaining to Modification 3. FIG. 28A, 28B are schematic diagrams illustrating ultrasound beam propagation paths pertaining to transmission according to the transmitter 103 of an ultrasound diagnostic device pertaining to the present disclosure, when a transmission focal point depth is less than a defined value.

As illustrated in FIG. 25, according to the ultrasound diagnostic device pertaining to Modification 3, when depth of the transmission focal point FP is “2”, the transmitter 103 selects the arrays Tx3, Tx2 of transmission transducers from the transducers 101a, then supplies drive signals pw3, pw2 having frequency distributions corresponding to “A, D” to the arrays Tx3, Tx2. Then, as illustrated in FIG. 28A, the ultrasound beams UsIn, UsO3 focused on the transmission focal point FP are transmitted from the arrays Tx3, Tx2, respectively.

Further, as illustrated in FIG. 25, according to the ultrasound diagnostic device pertaining to Modification 3, when depth of the transmission focal point is “1”, the transmitter 103 selects on the array Tx3 of transmission transducers from the transducers 101a, and supplies the drive signal pw3 with a frequency distribution corresponding to “A” to the array Tx3. As illustrated in FIG. 28B, only the ultrasound beam UsIn focused on the transmission focal point FP is transmitted from the array Tx3 of transmission transducers.

More specifically, when depth of the transmission focal point FP is equal to or less than a defined value, a total number of transmission transducers to be included in the array Tx of transmission transducers is preferably reduced to enable focusing of ultrasound beams to the transmission focal point FP. For example, in the example illustrated in FIG. 25, when depth of the transmission focal point FP is “2”, the total number of transmission transducers is 18, and when depth of the transmission focal point FP is “1”, the total number of transmission transducers is 8. A minimum value of the number of transducers for forming ultrasound beams UsIn, UsO3 is 8 ((64× 1/16)×2), and therefore when the depth of the transmission focal point FP is “2”, 2 types of ultrasound beam can be transmitted and UsIn and UsO3 containing high-frequency fundamental wave components are selected. When the depth of the transmission focal point FP is “1”, only one type of ultrasound beam can be transmitted and only UsIn containing a high-frequency fundamental wave component is selected. By changing assignment of transducer group according to the number of transmission transducers set for each transmission focal point in this way, even when depth of the transmission focal point FP is equal to or less than a defined value and the number of transmission transducers is small, an ultrasound beam including high-frequency fundamental wave components can be formed, ensuring depth of a transmitted wave.

### Modification 4

According to the ultrasound diagnostic device pertaining to Embodiments 1 and 2, the transmitter 103 selects a tertiary array Tx3 of transducers whose position in the azimuth direction overlaps with the position in the azimuth direction of the transmission focal point, two partial primary arrays Tx1 of transducers that sandwich the tertiary array Tx3 in the azimuth direction, and two secondary arrays Tx2 of transducers that sandwich the transducers of the tertiary array Tx3 and the partial primary arrays Tx1 in the azimuth direction. Further, the transmitter 103 causes transmission of an ultrasound beam having a higher signal intensity in a high frequency band from the tertiary array Tx3 and the secondary arrays Tx2 than from the partial primary arrays Tx1.

However, the transmitter 103 may select a single continuous transducer array as a primary transducer array Tx1 without splitting the array into two partial primary transducer arrays Tx1. In this case, the secondary transducer arrays Tx2 sandwich the primary transducer array Tx1 and the tertiary transducer array Tx3 is not used.

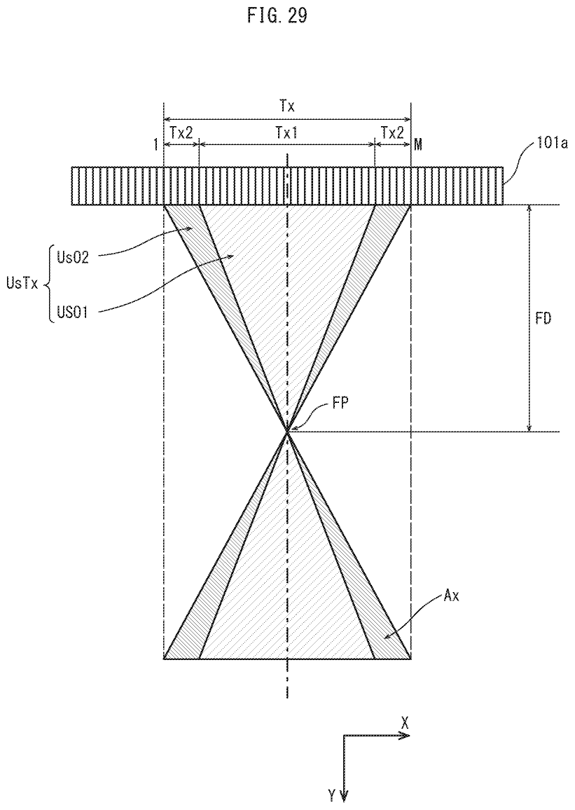

FIG. 29 is a schematic diagram illustrating a propagation path of an ultrasound beam pertaining to transmission by the transmitter of an ultrasound diagnostic device according to Modification 4. As illustrated in FIG. 29, according to the ultrasound diagnostic device of Modification 4, the transmitter 103 causes the ultrasound beam UsO1 to be transmitted from the primary transducer array Tx1 and the ultrasound beam UsO2 to be transmitted from the secondary transducer arrays Tx2, where the ultrasound beam UsO2 has a higher signal intensity in a high frequency band than the ultrasound beam UsO1. The absence of the tertiary transducer array Tx3 from which an ultrasound beam with a higher signal intensity in a high frequency band than the ultrasound beam from the primary transducer array Tx1 is a point of difference from Embodiments 1 and 2.

According to Modification 4, a reflected wave from an observation point Pij can be received from a wide viewing angle at a reception aperture Rxq corresponding to a reception steering angle θRC of a calculation target area Bxq, when irradiated with the ultrasound beam UsO2 that has a high signal intensity in a high frequency band from the secondary transducer arrays Tx2. Thus, a probability that a reflected wave from an anisotropic reflection site in a shallow region or peripheral region thereof can be received by any one of the reception apertures Rx of the calculation target areas Bxq is increased, and can be received by the most sensitive of the reception apertures Rxq.

### Modification 5

According to the ultrasound diagnostic device pertaining to Embodiments 1 and 2, the transducers 101a of the probe 101 are arranged in the azimuth direction. However, shape of the probe 101 may be, for example, a linear probe or convex probe.

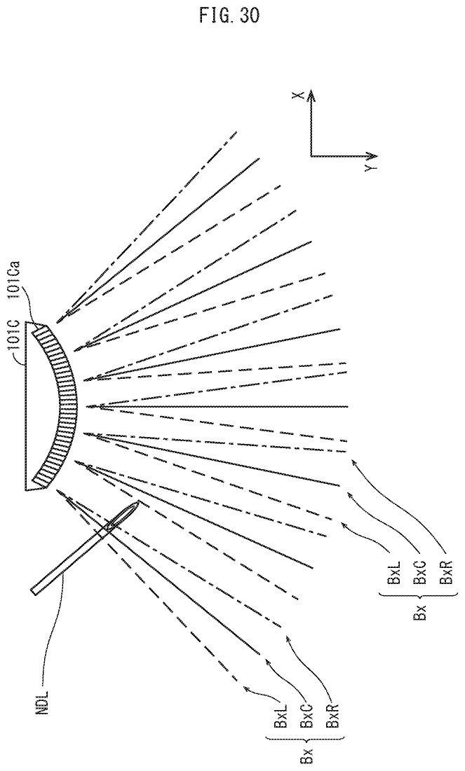

FIG. 30 is a schematic diagram illustrating ultrasound beam propagation paths pertaining to transmission according to the transmitter 103 of an ultrasound diagnostic device pertaining to Modification 5. As illustrated in FIG. 30, according to the ultrasound diagnostic device pertaining to Modification 5, a convex probe 101C includes transducers 101Ca arrayed on a convex surface. When a direction perpendicular to the azimuth direction is defined as a depth direction, length in the depth direction of a calculation target area BxC having a small angle between the depth direction and extension directions of scanning lines parallel to an area center line is equivalent to length in the depth direction of calculation target areas BxL, BxR that have a larger angle.

Convex probes have a measurement range in the depth direction of 20 cm to 30 cm, which is longer than linear probes. For this reason, frame rate reduction is required for convex probes.

According to the ultrasound diagnostic device pertaining to Modification 5, the length in the depth direction of the calculation target area Bx is ensured regardless of the angle between the scanning line extension direction and the depth direction, and therefore measurement of a deeper region is possible. Thus, Modification 5 is adaptable to the properties of convex probes, for which the measurement range in the depth direction is large.

Further, according to the ultrasound diagnostic device pertaining to Modification 5, the transmission steering angle θT may be a single direction. Thus, Modification 5 is adaptable to the properties of convex probes, for which frame rate needs to be reduced.

### Other Modifications

(1) Configurations of the transmitter 103 and the receiver 104 can be appropriately changed from the configurations described above.

For example, according to the ultrasound diagnostic device pertaining to Embodiments 1 and 2, the delay-and-sum unit executes delay-and-sum processing with respect to observation points in calculation target areas to generate acoustic line signal sub-frame data, and the imaging signal synthesizer synthesizes signals based on generated acoustic line signal sub-frame data using observation point positions as a reference, in order to generate ultrasound imaging signal frame data. In contrast, an ultrasound diagnostic device pertaining to a modification may store signals related to calculation target areas obtained from delay-and-sum processing by the delay-and-sum unit in a memory such as the image memory 106a, and generate one frame of frame data based on the signals stored. In other words, ultrasound imaging signal frame data may be generated without going through a process of generating sub-frame data as a data set corresponding to calculation target areas.

(2) The transmitter 103 according to at least one embodiment may set the array Tx of transmission transducers to be a portion of the transducers 101a of the probe 101, and repeatedly cause ultrasound transmission while gradually moving the array Tx in the array direction, and may set the array Tx of transmission transducers to be all of the transducers 101a of the probe 101, and cause ultrasound transmission all of the transducers 101a of the probe 101.

(3) The calculation target area Bx is not limited to a rectangular area, and may be an area having another shape, such as a trapezoid or arc. Further, the number of calculation target areas Bx is not limited to 3, and may be 2, 4, 6, 7, or larger numbers, and the calculation target areas Bx are not limited to the three types of L, C, R. Further, the calculation target area Bx is not limited to being symmetrical about a center of a transducer array. Further, the calculation target area Bx may be an hourglass-shaped area similar to the ultrasound irradiation area Ax. Further, the calculation target areas Bx may be set each transmission event to overlap in the transducer array direction. By synthesizing acoustic line signals of overlapping areas by a synthetic aperture method, it is possible to improve signal-to-noise ratio of an ultrasound image.

(4) Steering angles pertaining to transmission and reception are of course not limited to −10°, 0°, +10°. −30°, 0°, +30°, and other angle combinations may be used. Further, a steering angle for transmission and a steering angle for reception may be different. Transmission of ultrasound beams at different steering angles is not limited to being performed sequentially, and may be performed simultaneously if the number of probe elements and the number of system channels are sufficient.

(5) The number of transducers 101a can be set arbitrarily. Further, a linear scanning type electronic scanning probe may be used, an electronic scanning type or a mechanical scanning type may be adopted, and any one of a linear scanning type, a sector scanning type, and a convex scanning type may be adopted.

(6) The present disclosure is based on the embodiments above, but the present disclosure is not limited to these embodiments, and the following examples are also included in the scope of the present disclosure.

For example, the present disclosure may include a computer system including a microprocessor and a memory, the memory storing a computer program and the microprocessor operating according to the computer program. For example, the present disclosure may include a computer system that operates (or instructs operation of connected elements) according to a computer program of a diagnostic method of an ultrasound diagnostic device of the present disclosure.

Further, examples in which all or part of the ultrasound diagnostic device, or all or part of a beamforming section are constituted by a computer system including a microprocessor, a storage medium such as read-only memory (ROM), random-access memory (RAM), etc., a hard disk unit, and the like, are included in the present disclosure. A computer program for achieving the same operations as the devices described above may be stored in RAM or a hard disk unit. The microprocessor operating according to the computer program, thereby realizing the functions of each device.

Further, all or part of the elements of each device may be configured as one system large scale integration (LSI). A system LSI is an ultra-multifunctional LSI manufactured by integrating a plurality of elements on one chip, and more specifically is a computer system including a microprocessor, ROM, RAM, and the like. The plurality of elements can be integrated on one chip, or a portion may be integrated on one chip. Here, LSI may refer to an integrated circuit, a system LSI, a super LSI, or an ultra LSI, depending on the level of integration. A computer program for achieving the same operation as the devices described above may be stored in the RAM. The microprocessor operates according to the computer program, the system LSI thereby realizing the functions. For example, a case of the beamforming method of the present disclosure stored as a program of an LSI, the LSI inserted into a computer, and a defined program (beamforming method) being executed is also included in the present disclosure.

Note that methods of circuit integration are not limited to LSI, and implementation may be achieved by a dedicated circuit or general-purpose processor. After LSI manufacture, a field programmable gate array (FPGA) or a reconfigurable processor, in which circuit cell connections and settings in the LSI can be reconfigured, may be used.

Further, if a circuit integration technology is introduced that replaces LSI due to advances in semiconductor technology or another derivative technology, such technology may of course be used to integrate the function blocks.

Further, all or part of the functions of an ultrasonic diagnostic device pertaining to at least one embodiment may be implemented by execution of a program by a processor such as a CPU. All or part of the functions of an ultrasound diagnostic device pertaining to at least one embodiment may be implemented by a non-transitory computer-readable storage medium on which a program is stored that causes execution of a diagnostic method or beamforming method of an ultrasound diagnostic device described above. A program and signals may be recorded and transferred on a storage medium so that the program may be executed by another independent computer system, or the program may of course be distributed via a transmission medium such as the Internet.

Alternatively, elements of the ultrasound diagnostic device pertaining to at least one embodiment may be implemented by a programmable device such as a CPU, a GPU, a processor, or the like, and software. This may be referred to as general-purpose computing on a graphics processing unit (GPGPU). These components can each be a single circuit component or an assembly of circuit components. Further, a plurality of components can be combined into a single circuit component or can be an aggregate of a plurality of circuit components.

According to the ultrasound diagnostic device pertaining to at least one embodiment, the ultrasound diagnostic device includes a data storage as a storage device. However, the storage device is not limited to this example and a semiconductor memory, hard disk drive, optical disk drive, magnetic storage device, or the like may be externally connectable to the ultrasound diagnostic device.

Further, the division of function blocks in the block diagrams is merely an example, and a plurality of function blocks may be implemented as one function block, one function block may be divided into a plurality, and a portion of a function may be transferred to another function block. Further, a single hardware or software element may process the functions of a plurality of function blocks having similar functions in parallel or by time division.

Further, the order in which steps described above are executed is for illustrative purposes, and the steps may be in an order other than described above. Further, a portion of the steps described above may be executed simultaneously (in parallel) with another step.

Further, the ultrasound diagnostic device is described as having an externally connected probe and display, but may be configured with an integral probe and/or display.

Further, a portion of functions of transmitters and receivers may be included in the probe. For example, a transmission electrical signal may be generated and converted to ultrasound in the probe, based on a control signal for generating a transmission electrical signal outputted from the transmitter. It is possible to use a structure that converts received reflected ultrasound into a reception electrical signal and generates a reception signal based on the reception electrical signal in the probe.

Further, at least a portion of functions of each ultrasound diagnostic device pertaining to an embodiment, and each modification thereof, may be combined. Further, the numbers used above are all illustrative, for the purpose of explaining the present invention in detail, and the present disclosure is not limited to the example numbers used above.

Further, the present disclosure includes various modifications that are within the scope of conceivable ideas by a person skilled in the art.

### <<Review>>

As described above, the ultrasound diagnostic device pertaining to at least one embodiment is an ultrasound diagnostic device that transmits an ultrasound beam into a subject using an ultrasound probe in which transducers are arrayed along an azimuth direction, and generates acoustic line signals based on reflected waves obtained from the subject, the ultrasound diagnostic device comprising: a transmitter that determines a transmission focal point corresponding to an ultrasound beam focal point, selects an array of transmission transducers from the transducers, and causes transmission of an ultrasound beam focused on the transmission focal point from the array of transmission transducers; an input unit that generates sequences of received signals corresponding one-to-one with reception transducers in an array selected from the transducers, based on reflected waves received by the array of reception transducers; a delay-and-sum unit that determines, from analysis target ranges in the subject, calculation target areas that partially overlap each other, selects a reception aperture transducer array from the reception transducers, and with respect to observation points in the calculation target areas, executes delay-and-sum processing of the received signal sequences corresponding one-to-one with the reception transducers included in the reception aperture; and an imaging signal synthesizer that synthesizes results of the delay-and-sum processing using positions of the observation points for reference to generate ultrasound imaging signal frame data, wherein the transmitter selects, as the array of transmission transducers, a primary transducer array and two secondary transducer arrays that sandwich the first transducer array in the azimuth direction, a portion of the ultrasound beam from the secondary transducer arrays has a larger signal intensity in a high frequency band than a portion of the ultrasound beam from the primary transducer array, and the calculation target areas each have a different position along the azimuth direction.

As a result, in an inexpensive device that does not require complex transmission control, visibility of an anisotropic high-reflection member in a shallow peripheral region of ultrasound irradiation is improved, and rendering of a high-angle anisotropic reflection site can be improved over conventional technology.

According to at least one embodiment, the primary transducer array is composed of a plurality of partial primary transducer arrays that are separated from each other in the azimuth direction, and the transmitter further selects, as the array of transmission transducers, a tertiary transducer array sandwiched between the partial primary transducer arrays, and a portion of the ultrasound beam from the tertiary transducer array has a larger signal intensity in the high frequency band than the portion of the ultrasound beam from the primary transducer array.

According to at least one embodiment, when depth of the transmission focal point is greater than or equal to a defined value, the transmitter selects, as the array of transmission transducers, the tertiary transducer array, the partial primary transducer arrays, and the secondary transducer arrays.

According to at least one embodiment, the results of the delay-and-sum processing are acoustic line signal sub-frame data, and the acoustic line signal sub-frame data is synthesized to generate the ultrasound imaging signal frame data.

According to this structure, while an ultrasound beam UsO2 having a large signal intensity in a high frequency band is emitted from the arrays Tx3, reflected waves from observation points Pij are received from a wide viewing angle by a reception aperture Rxq corresponding to a reception steering angle ORC of a calculation target area Bxq in an ultrasound irradiation area Ax in a shallow region where attenuation of a high frequency band is small. Therefore, for example, reflected waves from an anisotropic reflection site located in a shallow region or a peripheral region thereof, such as a high-angle puncture needle shaft, a longitudinal tissue boundary, an anterior talofibular ligament, or the like, can be received with higher probability at any of the reception apertures Rx of the calculation target areas Bxq, and reflected waves can be received by the reception aperture Rxq with the highest sensitivity.

As a result, in an inexpensive device that does not require complex transmission control, visibility of an anisotropic high-reflection member in a shallow peripheral region of ultrasound irradiation is improved, and rendering of a high-angle anisotropic reflection site can be improved over conventional technology.

Further, according to at least one embodiment, when a transmission steering angle is a reference angle and an angle between a direction indicating the reference angle and a center line of a calculation target area is defined as a first shift angle, among the calculation target areas, the delay-and-sum unit sets depth in a depth direction for a calculation target area for which the first shift angle is large to be shorter than depth in the depth direction for a calculation target area for which the first shift angle is small.

According to this structure, even if a calculation target area Bx with a large reception steering angle θR is enlarged to a deeper region, the calculation target area Bx does not become an image display region, and a disadvantage of expanding a reception steering angle θR wherein it is difficult to obtain a good signal to noise ratio because attenuation is high when a propagation path is long is reduced. Accordingly, resources for calculation can be efficiently used to effectively expand a viewing angle, and reflected waves from anisotropic high-reflection members that are common in shallow regions can be efficiently received, increasing visibility thereof.

Further, according to at least one embodiment, in the azimuth direction, an array center of the reception aperture in the delay-and-sum processing is within the tertiary transducer array when an observation point is located in an area between two straight lines intersecting the transmission focal point and ends of the tertiary transducer array, and within one of the secondary transducer arrays when an observation point is located in an area between two straight lines intersecting the transmission focal point and ends of the one of secondary transducer arrays.

According to this structure, a viewing angle for received signals in delay-and-sum processing can be increased in proportion to an increase in reception steering angle θR.

Further, according to at least one embodiment, when a transmission steering angle is a reference angle, the delay-and-sum unit sets an array center of the reception aperture relative to an observation point such that a reception direction angle is identical to the reference angle.

According to this structure, a reflected wave from an observation point Pij can be received with highest sensitivity to the observation point Pij, and artifacts due to grating lobes can be reduced, improving signal to noise ratio.

Further, according to at least one embodiment, the transmitter further selects two quaternary transducer arrays between the primary transducer array and the secondary transducer arrays or between the partial primary transducer arrays and the tertiary transducer array, from which the ultrasound beam is not transmitted.

According to this structure, width in the azimuth direction of an entire array of transmission transducers Tx can be increased while suppressing an increase in energy consumption due to application of the drive signal pw, and an absolute value of a viewing angle of reception signals in delay-and-sum processing can be increased.

Further, according to at least one embodiment, a portion of the ultrasound beam from the secondary transducer arrays has a larger signal intensity in a high frequency band than a portion of the ultrasound beam from the tertiary transducer array and a smaller signal intensity in a low frequency band than a portion of the ultrasound beam from the tertiary transducer array and the partial primary transducer arrays.

According to this structure, a decrease in signal intensity of a harmonic component in the UsO3 area due to a decrease in directivity of the transducers 101a at a steering angle can be compensated for, and an occurrence of ultrasound beam splitting in a deeper region can be suppressed.

Further, according to at least one embodiment, a portion of the ultrasound beam from the secondary transducer arrays has a larger signal intensity in a high frequency band than a portion of the ultrasound beam from the tertiary transducer array and a smaller signal intensity in a low frequency band than a portion of the ultrasound beam from the tertiary transducer array.

Further, according to at least one embodiment, when depth of the transmission focal point is less than a defined value, the transmitter selects, as the array of transmission transducers, the tertiary transducer array, and the ultrasound beam includes a high frequency band greater than or equal to a defined value.

According to this structure, even when depth of the transmission focal point FP is less than a defined value, an ultrasound beam including sufficient high-frequency fundamental component can be formed, increasing resolution in a shallow region.

Further, according to at least one embodiment, when a direction perpendicular to a tangent direction of the array of transducers is defined as a depth direction and an angle between the depth direction and an extension direction of a scan line parallel to a center line of a calculation target area is defined as a second shift angle, among the calculation target areas, depth of a calculation target area for which the second shift angle is small is equivalent to depth of a calculation target area for which the second shift angle is large.

According to this structure, length of a calculation target area Bx in the depth direction is ensured and measurement to a deeper region is made possible regardless of an angle of a direction of extension of a scan line relative to the depth direction, and the embodiment can be adapted to the properties of convex probes that have a large measurement range in the depth direction. In addition, convex probes typically require frame rate reduction, but the embodiment can be adapted to this characteristic by setting the transmission steering angle θT to a single direction.

Further, according to at least one embodiment, the transmitter sets the transmission focal point multiple times, each time with a different position in the azimuth direction, and causes transmission of the ultrasound beam multiple times, each time focused on a different one of the transmission focal points, and the imaging signal synthesizer, when generating the ultrasound imaging signal frame data corresponding to the transmission focal points, further synthesizes the ultrasound imaging signal frame data based on positions of observation points to generate ultrasound imaging signal integrated frame data.

According to this structure, absolute values of viewing angles and number of directions in delay-and-sum processing are increased, probability of capture by any reception aperture Rxq of a reflected wave reflected from an anisotropic high reflection member in a shallow region or peripheral region thereof is increased, increasing visibility of anisotropic high reflection members.

According to at least one embodiment, the transmitter moves the selection of the array of transmission transducers in the azimuth direction gradually, while setting the transmission focal point in the azimuth direction according to the selection, thereby causing the transmission of the ultrasound beam from each of the selections of array of transmission transducers focused to each of the transmission focal points set, and the imaging signal synthesizer, when generating the ultrasound imaging signal frame data corresponding to the arrays of transmission transducers and the transmission focal points, further synthesizes the ultrasound imaging signal frame data based on positions of observation points to generate ultrasound imaging signal integrated frame data.

According to this structure, for the same observation point, an ultrasound imaging signal can be generated based on reception signals from different positions of the array Tx of transmission transducers, and spatial resolution and signal to noise ratio can be improved. Further, when an anisotropic specular reflection member is located in a shallow region farther outwards than either end of the transducers 101a of the probe 101, a reflected wave from the anisotropic specular reflection member is effectively received, making it possible to improve visibility of the anisotropic specular reflection member such as a puncture needle.

According to at least one embodiment, the ultrasound diagnostic device further includes an image generator, the transmitter causes transmission of a pair of ultrasound waves whose polarity is inverted on a same scan line, the input unit generates a reception signal sequence based on a pair of reflected waves based on the pair of ultrasound waves, and the image generator extracts harmonic components from the reception signal sequence based on the pair of reflected waves, and generates an ultrasound imaging signal based on the harmonic components.

According to this structure, sufficient spatial resolution and signal to noise ratio can be obtained by using tissue harmonic imaging (THI) that extracts a harmonic component where a fundamental wave is sufficiently eliminated by pulse inversion applied to acoustic line signals.

Further, according to at least one embodiment, the transmitter supplies a drive signal such that, in a frequency band included in a −20 dB transducer transmission frequency band, frequency distribution with respect to at least one of the tertiary transmission transducer array and the secondary transducer arrays has intensity peaks at a lower and a higher frequency than a center frequency of the −20 dB transducer transmission frequency band, and intensity in a frequency band between these intensity peaks is −20 dB or more, using a maximum value of the intensity peaks as a reference.

According to this structure, a high-frequency fundamental wave component is focused by an acoustic lens to generate a harmonic component from a shallow region, so that it is possible to achieve high resolution in visualization of the shallow region, as well as effectively receiving reflected waves from an anisotropic reflection member as commonly found in shallow regions, improving visibility of specular reflection members such as a puncture needle. Further, sound pressure that can generate a harmonic at a transmission focal point can be formed by a low-frequency fundamental wave component, and therefore a harmonic image with a good signal to noise ratio over a wide depth range from the shallow region to the transmission focal point can be obtained.

Further, according to at least one embodiment, the transmitter supplies a drive signal such that, in a frequency band included in a −20 dB transducer transmission frequency band, frequency distribution with respect to the partial primary transmission transducer arrays has a maximum intensity peak at a lower frequency than a center frequency of the −20 dB transducer transmission frequency band.

According to this structure, harmonic generation in a shallow region in the UsO1 area is suppressed, acoustic noise in the UsIn, UsO2, UsO3 areas in which observation points Pij are set is suppressed, while also transmitting the ultrasound beam UsO1 including low heat, low attenuation low-frequency band signal intensity from the arrays Tx2, and thereby improving depth of penetration and efficiently using applied energy.

Further, according to at least one embodiment, a −20 dB frequency band drive signal supplied by the transmitter to the tertiary transmission transducer array and the secondary transmission transducer arrays is wider than a −20 dB frequency band drive signal supplied by the transmitter to the partial primary transmission transducer arrays.

Further, a control method of an ultrasound diagnostic device pertaining to at least one embodiment is a control method of an ultrasound diagnostic device that transmits an ultrasound beam into a subject using an ultrasound probe in which transducers are arrayed along an azimuth direction, and generates acoustic line signals based on reflected waves obtained from the subject, the control method comprising: determining a transmission focal point corresponding to an ultrasound beam focal point, selecting an array of transmission transducers from the transducers, and causing transmission of an ultrasound beam focused on the transmission focal point from the array of transmission transducers; generating sequences of received signals corresponding one-to-one with reception transducers in an array selected from the transducers, based on reflected waves received by the array of reception transducers; determining, from analysis target areas in the subject, calculation target areas that partially overlap each other, selecting a reception aperture transducer array from the reception transducers, and with respect to observation points in the calculation target areas, executing delay-and-sum processing of the received signal sequences corresponding one-to-one with the reception transducers included in the reception aperture; and synthesizing results of the delay-and-sum processing using positions of the observation points for reference to generate ultrasound imaging signal frame data, wherein a primary transducer array and two secondary transducer arrays that sandwich the primary transducer array in the azimuth direction are selected as the array of transmission transducers, a portion of the ultrasound beam from the secondary transducer arrays has a larger signal intensity in a high frequency band than a portion of the ultrasound beam from the primary transducer array, and the calculation target areas each have a different position along the azimuth direction.

According to this configuration, in an inexpensive device that does not require complex transmission control, visibility of an anisotropic high-reflection member in a shallow peripheral region of ultrasound irradiation is improved, and rendering of a high-angle anisotropic reflection site can be improved over conventional technology.

Further, according to at least one embodiment, the primary transducer array is composed of a plurality of partial primary transducer arrays that are separated from each other in the azimuth direction, and a tertiary transducer array sandwiched between the partial primary transducer arrays is further selected as part of the array of transmission transducers, and a portion of the ultrasound beam from the tertiary transducer array has a larger signal intensity in the high frequency band than the portion of the ultrasound beam from the primary transducer array.

Further, according to at least one embodiment, when depth of the transmission focal point is greater than or equal to a defined value, the tertiary transducer array, the partial primary transducer arrays, and the secondary transducer arrays are selected as the array of transmission transducers.

Further, according to at least one embodiment, the results of the delay-and-sum processing are acoustic line signal sub-frame data, and the acoustic line signal sub-frame data is synthesized to generate the ultrasound imaging signal frame data.

According to this configuration, in an inexpensive device that does not require complex transmission control, visibility of an anisotropic high-reflection member in a shallow peripheral region of ultrasound irradiation is improved, and rendering of a high-angle anisotropic reflection site can be improved over conventional technology.

### <<Supplement>>

The embodiments described above each indicate one preferred specific example of the present disclosure. Numerical values, shapes, materials, constituent elements, arrangement positions and connections of constituent elements, steps, order of steps, and the like indicated as embodiments are merely examples and are not intended to limit the present disclosure. Further, among constituent elements in the embodiments, elements not described in independent claims representing top level concepts of the present disclosure are described as any constituent element constituting a more beneficial embodiment.

Further, the order in which steps described above are executed is for illustrative purposes, and the steps may be in an order other than described above. Further, a portion of the steps described above may be executed simultaneously (in parallel) with another step.

Further, in order to facilitate understanding, constituent elements in each drawing referenced by description of an embodiment are not necessarily to scale. Further, the present disclosure is not limited by the description of each embodiment, and can be appropriately changed without departing from the scope of the present disclosure.

Although the technology pertaining to the present disclosure has been fully described by way of examples with reference to the accompanying drawings, various changes and modifications will be apparent to those skilled in the art. Therefore, unless such changes and modifications depart from the scope of the present disclosure, they should be construed as being included therein.

What is claimed is:

1. An ultrasound diagnostic device that transmits an ultrasound beam into a subject using an ultrasound probe in which transducers are arrayed along an azimuth direction, and generates acoustic line signals based on reflected waves obtained from the subject, the ultrasound diagnostic device comprising:
ultrasound signal processing circuitry, the ultrasound signal processing circuitry comprising:
a transmitter that determines a transmission focal point corresponding to an ultrasound beam focal point, selects an array of transmission transducers from the transducers, and causes transmission of an ultrasound beam focused on the transmission focal point from the array of transmission transducers;
an input unit that generates sequences of received signals corresponding one-to-one with reception transducers in an array selected from the transducers, based on reflected waves received by the array of reception transducers;
a delay-and-sum unit that determines, from analysis target areas in the subject, calculation target areas that partially overlap each other, selects a reception aperture transducer array from the reception transducers, and with respect to observation points in the calculation target areas, executes delay-and-sum processing of the received signal sequences corresponding one-to-one with the reception transducers included in the reception aperture; and
an imaging signal synthesizer that synthesizes results of the delay-and-sum processing using positions of the observation points for reference to generate ultrasound imaging signal frame data, wherein
the transmitter selects, as the array of transmission transducers, a primary transducer array and two secondary transducer arrays that sandwich the primary transducer array in the azimuth direction,
a portion of the ultrasound beam from the secondary transducer arrays has a larger signal intensity in a high frequency band than a portion of the ultrasound beam from the primary transducer array, and
the calculation target areas each have a different position along the azimuth direction.

2. The ultrasound diagnostic device of claim 1, wherein
the primary transducer array is composed of a plurality of partial primary transducer arrays that are separated from each other in the azimuth direction, and
the transmitter further selects, as the array of transmission transducers, a tertiary transducer array sandwiched between the partial primary transducer arrays, and
a portion of the ultrasound beam from the tertiary transducer array has a larger signal intensity in the high frequency band than the portion of the ultrasound beam from the primary transducer array.

3. The ultrasound diagnostic device of claim 2, wherein
when depth of the transmission focal point is greater than or equal to a defined value, the transmitter selects, as the array of transmission transducers, the tertiary transducer array, the partial primary transducer arrays, and the secondary transducer arrays.

4. The ultrasound diagnostic device of claim 2, wherein
the results of the delay-and-sum processing are acoustic line signal sub-frame data, and
the acoustic line signal sub-frame data is synthesized to generate the ultrasound imaging signal frame data.

5. The ultrasound diagnostic device of claim 2, wherein
when a transmission steering angle is a reference angle and an angle between a direction indicating the reference angle and a center line of a calculation target area is defined as a first shift angle,
among the calculation target areas, the delay-and-sum unit sets depth in a depth direction for a calculation target area for which the first shift angle is large to be shorter than depth in the depth direction for a calculation target area for which the first shift angle is small.

6. The ultrasound diagnostic device of claim 2, wherein
in the azimuth direction, an array center of the reception aperture in the delay-and-sum processing is within the tertiary transducer array when an observation point is located in an area between two straight lines intersecting the transmission focal point and ends of the tertiary transducer array, and within one of the secondary transducer arrays when an observation point is located in an area between two straight lines intersecting the transmission focal point and ends of the one of secondary transducer arrays.

7. The ultrasound diagnostic device of claim 2, wherein
when a transmission steering angle is a reference angle, the delay-and-sum unit sets an array center of the reception aperture relative to an observation point such that a reception direction angle is identical to the reference angle.

8. The ultrasound diagnostic device of claim 2, wherein
the transmitter further selects two quaternary transducer arrays between the primary transducer array and the secondary transducer arrays or between the partial primary transducer arrays and the tertiary transducer array, from which the ultrasound beam is not transmitted.

9. The ultrasound diagnostic device of claim 2, wherein
a portion of the ultrasound beam from the secondary transducer arrays has a larger signal intensity in a high frequency band than a portion of the ultrasound beam from the tertiary transducer array and a smaller signal intensity in a low frequency band than a portion of the ultrasound beam from the tertiary transducer array and the partial primary transducer arrays.

10. The ultrasound diagnostic device of claim 2, wherein
a portion of the ultrasound beam from the secondary transducer arrays has a larger signal intensity in a high frequency band than a portion of the ultrasound beam from the tertiary transducer array and a smaller signal intensity in a low frequency band than a portion of the ultrasound beam from the tertiary transducer array.

11. The ultrasound diagnostic device of claim 2, wherein
when depth of the transmission focal point is less than a defined value, the transmitter selects, as the array of transmission transducers, the tertiary transducer array, and the ultrasound beam includes a high frequency band greater than or equal to a defined value.

12. The ultrasound diagnostic device of claim 2, wherein
when a direction perpendicular to a tangent direction of the array of transducers is defined as a depth direction and an angle between the depth direction and an extension direction of a scan line parallel to a center line of a calculation target area is defined as a second shift angle, among the calculation target areas, depth of a calculation target area for which the second shift angle is small is equivalent to depth of a calculation target area for which the second shift angle is large.

13. The ultrasound diagnostic device of claim 2, wherein
the transmitter sets the transmission focal point multiple times, each time with a different position in the azimuth direction, and causes transmission of the ultrasound beam multiple times, each time focused on a different one of the transmission focal points, and
the imaging signal synthesizer, when generating the ultrasound imaging signal frame data corresponding to the transmission focal points, further synthesizes the ultrasound imaging signal frame data based on positions of observation points to generate ultrasound imaging signal integrated frame data.

14. The ultrasound diagnostic device of claim 2, wherein
the transmitter moves the selection of the array of transmission transducers in the azimuth direction gradually, while setting the transmission focal point in the azimuth direction according to the selection, thereby causing the transmission of the ultrasound beam from each of the selections of array of transmission transducers focused to each of the transmission focal points set, and
the imaging signal synthesizer, when generating the ultrasound imaging signal frame data corresponding to the arrays of transmission transducers and the transmission focal points, further synthesizes the ultrasound imaging signal frame data based on positions of observation points to generate ultrasound imaging signal integrated frame data.

15. The ultrasound diagnostic device of claim 2, wherein
the delay-and-sum unit performs the delay-and-sum processing by calculating delay times of reflected waves reaching each of the transducers included in the reception aperture from observation points in calculation target areas, specifying portions of reception signal sequences corresponding to each transducer, based on the reflected waves from the observation points and the delay times, and summing

16. The ultrasound diagnostic device of claim 2, further comprising:
an image generator, wherein
the transmitter causes transmission of a pair of ultrasound waves whose polarity is inverted on a same scan line,
the input unit generates a reception signal sequence based on a pair of reflected waves based on the pair of ultrasound waves, and
the image generator extracts harmonic components from the reception signal sequence based on the pair of reflected waves, and generates an ultrasound imaging signal based on the harmonic components.

17. The ultrasound diagnostic device of claim 2, wherein
the transmitter supplies a drive signal such that, in a frequency band included in a −20 dB transducer transmission frequency band, frequency distribution with respect to at least one of the tertiary transmission transducer array and the secondary transducer arrays has intensity peaks at a lower and a higher frequency than a center frequency of the −20 dB transducer transmission frequency band, and intensity in a frequency band between these intensity peaks is −20 dB or more, using a maximum value of the intensity peaks as a reference.

18. The ultrasound diagnostic device of claim 2, wherein
the transmitter supplies a drive signal such that, in a frequency band included in a −20 dB transducer transmission frequency band, frequency distribution with respect to the partial primary transmission transducer arrays has a maximum intensity peak at a lower frequency than a center frequency of the −20 dB transducer transmission frequency band.

19. The ultrasound diagnostic device of claim 2, wherein
a −20 dB frequency band drive signal supplied by the transmitter to the tertiary transmission transducer array and the secondary transmission transducer arrays is wider than a −20 dB frequency band drive signal supplied by the transmitter to the partial primary transmission transducer arrays.

20. A control method of an ultrasound diagnostic device that transmits an ultrasound beam into a subject using an ultrasound probe in which transducers are arrayed along an azimuth direction, and generates acoustic line signals based on reflected waves obtained from the subject, the control method comprising:
determining a transmission focal point corresponding to an ultrasound beam focal point, selecting an array of transmission transducers from the transducers, and causing transmission of an ultrasound beam focused on the transmission focal point from the array of transmission transducers;
generating sequences of received signals corresponding one-to-one with reception transducers in an array selected from the transducers, based on reflected waves received by the array of reception transducers;
determining, from analysis target areas in the subject, calculation target areas that partially overlap each other, selecting a reception aperture transducer array from the reception transducers, and with respect to observation points in the calculation target areas, executing delay-and-sum processing of the received signal sequences corresponding one-to-one with the reception transducers included in the reception aperture; and
synthesizing results of the delay-and-sum processing using positions of the observation points for reference to generate ultrasound imaging signal frame data, wherein
a primary transducer array and two secondary transducer arrays that sandwich the primary transducer array in the azimuth direction are selected as the array of transmission transducers,
a portion of the ultrasound beam from the secondary transducer arrays has a larger signal intensity in a high frequency band than a portion of the ultrasound beam from the primary transducer array, and
the calculation target areas each have a different position along the azimuth direction.
